(19) United States
(12) Patent Application Publication
(10) Pub. No.: US 2025/0134921 A1
(43) Pub. Date: May 1, 2025

(54) ACETYLATED SIALIC ACID GLYCOCLUSTERS AND THEIR USES FOR TREATING INFECTIOUS DISEASES

(71) Applicants: UNIVERSITÉ CATHOLIQUE DE LOUVAIN, Louvain-la-Neuve (BE); UNIVERSITÉ DE NAMUR, Namur (BE)

(72) Inventors: David ALSTEENS, Corroy-le-Grand (BE); Stéphane VINCENT, Hyon (BE)

(73) Assignees: UNIVERSITÉ CATHOLIQUE DE LOUVAIN, Louvain-la-Neuve (BE); UNIVERSITÉ DE NAMUR, Namur (BE)

(21) Appl. No.: 18/690,372

(22) PCT Filed: Sep. 9, 2022
(86) PCT No.: PCT/EP2022/075109

§ 371 Date: Mar. 8, 2024

(30) Foreign Application Priority Data
Sep. 10, 2021 (EP) 21195975.4

(51) Int. Cl. A61K 31/7135; A61K 31/7056; A61P 31/14; C07H 1/00; C07H 15/26; C07H 23/00

(52) U.S. Cl. CPC A61K 31/7135; A61K 31/7056; A61P 31/14; C07H 1/00; C07H 15/26; C07H 23/00

(57) ABSTRACT

A glycocluster including at least two acetylated-sialic acids covalently linked to a macrocycle, wherein the macrocycle is selected from porphyrins, pillararenes, calixarenes and fullerenes. Each acetylated-sialic acid may be independently selected from 4-O-acetylated-sialic acid, 7-O-acetylated-sialic acid, 8-O-acetylated-sialic acid and 9-O-acetylated-sialic acid. Also a glycocluster for use in the treatment and/or prevention of an infectious disease, such as a coronavirus infection.

### FIELD OF INVENTION

The present invention belongs to the field of the treatment of infectious diseases and relates to novel glycoclusters comprising a plurality of acetylated sialic acids for use in the treatment of infectious diseases, especially a SARS-CoV-2 infection.

### BACKGROUND OF INVENTION

Coronaviruses (CoVs) are viruses of the subfamily Orthocoronavirinae, in the family Coronaviridae. They are enveloped viruses with a positive-sense single-stranded ribonucleic acid (RNA) genome and a helical nucleocapsid. Their name is due to their distinctive morphology, namely a series of club-shaped spikes projecting from the surface of their envelope that gives them a crown-like appearance. Coronaviruses are also characterized by an unusually large RNA genome and a specific replication strategy. RNA viruses and especially coronaviruses are responsible for a wide range of respiratory, systemic, gastrointestinal and neurologic diseases in mammals as well as birds.

Coronaviruses were first identified in humans about 50 years ago in the United Kingdom and the United States. They were since generally considered as causing only mild respiratory infectious diseases, e.g., the common cold. At the beginning of the 20^{th} century, two highly pathogenic coronaviruses were first identified: severe acute respiratory syndrome coronavirus (SARS-CoV) and Middle East respiratory syndrome coronavirus (MERS-CoV). By contrast with previous coronavirus infections, SARS and MERS were severe respiratory diseases and accounted for hundreds of deaths. At the end of 2019, a new infectious respiratory illness outbroke in Wuhan (China), and the cause was identified in the end as a new human coronavirus SARS-CoV-2. The genome of SARS-CoV-2 shares about 80% identity with that of SARS-CoV and is about 96% identical to that of the bat coronavirus BatCoV RaTG13. The disease caused by SARS-COV-2 infection is named “coronavirus disease 2019” (“COVID-19”). COVID-19 quickly emerged as a severe world-scale pandemic in 2020.

It is considered that SARS-CoV-2 infection remains asymptomatic for a significant number of infected subjects. For symptomatic patients, COVID-19 presents generally (about 80%) as a respiratory disease of mild severity whose symptoms may include fever, cough or other respiratory symptoms (such as mild breath shortness or chest tightness), headache, fatigue or muscle pain, and loss of smell and taste. However, some patients (about 15%) develop severe symptoms including dyspnea, hypoxia or pneumonia (“COVID-19 pneumonia”). In a small number of cases (about 5%), critical symptoms such as respiratory failure, shock, or multiorgan failure are observed. It was estimated that about 20% of COVID-19 patients require hospitalization and some of them (about 5%) even have to be admitted to intensive care unit (ICU). Permanent damage to organs has been observed in some cases, and some patients continue to experience a range of effects even months after recovery (“long COVID”). Thus, COVID-19 causes substantial suffering and death, and also endangers many health systems in the world.

Several vaccines against SARS-CoV-2 have been approved and distributed in various countries. By contrast, although work is underway to develop drugs that inhibit the virus for prevention and/or treatment purposes, the primary treatment of COVID-19 remains symptomatic, as well as ancillary measures such as supportive care and isolation. Moreover, some mass vaccination campaigns face significant drawbacks in many countries, a lot of people are likely to be affected by COVID-19 in the nearby future.

Therefore, there is still a need for an effective treatment and/or prevention of coronavirus infections, in particular coronavirus respiratory infections causing diseases such as SARS, MERS or COVID-19. In particular, there is a need for therapeutic agents highly efficient against coronavirus replication, with few or no significant adverse effects, having good chemical stability and/or low cost.

SARS-CoV entry into host cells is mediated by its transmembrane spike (S) glycoprotein that forms homotrimers protruding from the viral surface. The S glycoprotein comprises two functional subunits responsible either for binding to the host cell receptor (S1 subunit including the receptor-binding domain (RBD)) or for fusion of the viral and cellular membranes (S2 subunit). Angiotensin-converting enzyme 2 (ACE2), previously identified as the cellular receptor for SARS-CoV, also acts as a receptor of the new coronavirus (SARS-CoV-2). In the case of SARS-CoV, the S glycoprotein on the virion surface mediates receptor recognition and membrane fusion. Recently, the high-resolution cryo-electron microscopy structure obtained on the full-length human ACE2 in the presence of the RBD of the S glycoprotein of SARS-CoV-2 suggested simultaneous binding of two S-glycoprotein trimers to an ACE2 dimer. The S2 subunit is further cleaved by host proteases located immediately upstream of the fusion peptide, leading to the activation of the glycoprotein that undergoes extensive irreversible conformational changes, which facilitates the membrane fusion process.

While S-protein binding to ACE2 has already been extensively studied and a consensus reached about its central role in infection, several studies suggest a pivotal role of co-receptors/attachment factors of other cell surface molecules. In particular, it was recently suggested that 9-O-acetyl-sialogycans may possibly be involved in the early stage of SARS-CoV-2 binding to cells, in addition to ACE2 (Yang, J. et al., Nature Communications, 2020, Vol. 11, Article number: 4541). The binding of an acetylated-sialic acid with coronavirus S glycoproteins and coronavirus hemagglutinin esterases has also be reported (Tortorici, M. A. et al., Nature Structural Molecular Biology, 2019, Vol. 26, pp. 481-489). Therefore, based on this preliminary information, it may be thought that sialic acid derivatives could have a potential to act as competitive inhibitors to block the interactions between SARS-CoV-2 spike protein S and host cells, whose interactions usually mediate the first step of infection (Tortorici, M. A. et al., Nature Structural Molecular Biology, 2019, Vol. 26, pp. 481-489).

However, although it is suggested that sialic acid and its acetylated derivatives could likely bind, at least weakly, the spike protein of SARS-CoV-2, there is no direct quantitative evidence to define which one of these two carbohydrates would be the optimal partner (Yang, J. et al., Nature Communications, 2020, Vol. 11, Article number: 4541; Nguyen, K. et al., Viruses, May 2021, Vol. 13, No. 5, p. 927).

Moreover, it is known in the art that individual glycans such as sialic acid or its derivatives generally have a relatively low affinity for their protein targets, typically moderate at best (Sauter, N. K. et al., Biochemistry 1989, Vol. 28, p. 8388; Dormitzer, P. R. et al., Journal of Virology, October 2002, Volume 76, No. 20, pp. 10512-10517). This is further evidenced in the Examples of the present application. The limited affinity of individual sialic acid and derivatives thereof renders such glycans unsuitable for direct use in actual treatment and/or prevention of viral infections.

Surprisingly, the Applicants evidenced that, when at least two sialic acids or derivatives thereof are linked to specific macrocycles (such as porphyrins, pillararenes, calixarenes and fullerenes), the obtained glycoclusters are potent competitors of the SARS-CoV-2 early attachment to the host cells, despite the low affinity of the sialic acid. Thus, in sharp contrast with sialic acids that are not linked to a macrocycle (“free” sialic acids), the glycoclusters of the invention are suitable for use in the treatment and/or prevention of an infectious disease such as COVID-19. The present invention thus opens the way to new medicaments against viral infections such as COVID-19.

### SUMMARY

This invention relates to a glycocluster comprising at least two acetylated-sialic acids covalently linked to a macrocycle, wherein said macrocycle is selected from porphyrins, pillararenes, calixarenes and fullerenes; and wherein each acetylated-sialic acid is independently selected from 4-O-acetylated-sialic acid, 7-O-acetylated-sialic acid, 8-O-acetylated-sialic acid and 9-O-acetylated-sialic acid.

According to one embodiment, the glycocluster comprises at least four acetylated-sialic acids covalently linked to said macrocycle. According to one embodiment, each acetylated-sialic acid is independently selected from 7-O-acetylated-sialic acid and 9-O-acetylated-sialic acid. In one embodiment, each acetylated-sialic acid is 9-O-acetylated-sialic acid.

According to one embodiment, the macrocycle is selected from porphyrins, preferably selected from [Zn(tetraphenylporphyrin)] and tetraphenylporphyrin. According to one embodiment, the glycocluster further comprises at least one angiotensin-converting enzyme 2 (ACE2) binding inhibitor; preferably the angiotensin-converting enzyme 2 (ACE2) binding inhibitor is selected from ACE2 binding inhibitor peptides, ACE2 binding inhibitor proteins and anti-ACE2 antibodies or antigen-binding fragments thereof.

According to one embodiment, the glycocluster is a compound of formula (I), (Ia), (II), (III) or (IV)

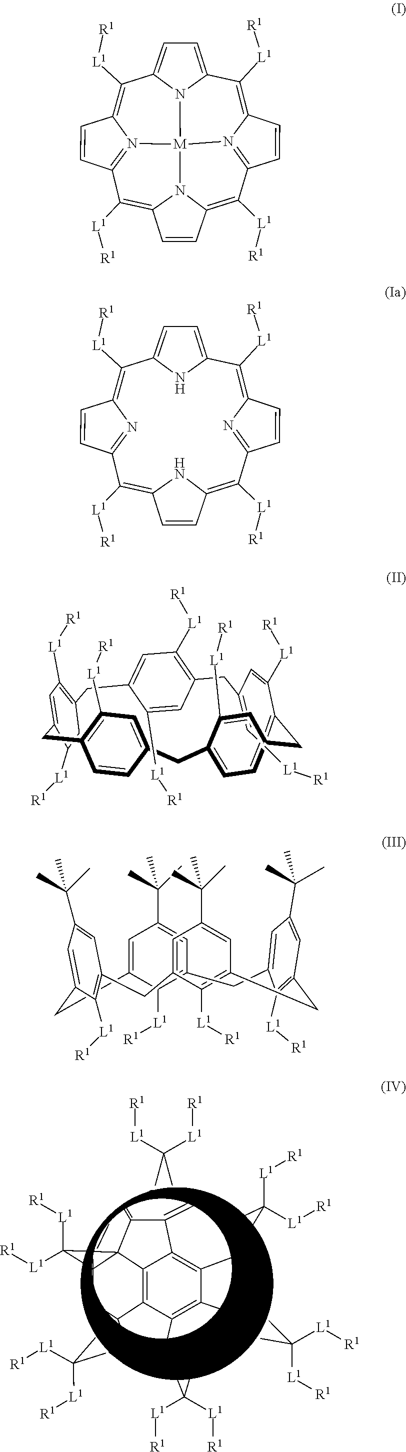

or a pharmaceutically acceptable salt and/or solvate thereof;
wherein each L¹ is independently: a single bond or a linker selected from alkyl, heteroalkyl, alkenyl, alkynyl, aryl, alkylaryl, arylalkyl, heteroaryl, heteroarylalkyl and alkylheteroaryl; wherein said alkyl, heteroalkyl, alkenyl, alkynyl, aryl, alkylaryl, arylalkyl, heteroaryl, heteroarylalkyl or alkylheteroaryl optionally comprises at least one coupling product; each R¹ is independently selected from acetylated-sialic acids and ACE2 binding inhibitors; provided that at least two R¹ are acetylated-sialic acids; and M is a metal cation.

According to one embodiment, each L¹ is a linker selected from alkyl, heteroalkyl, alkylaryl, arylalkyl, heteroarylalkyl and alkylheteroaryl; wherein said alkyl, heteroalkyl, alkylaryl, arylalkyl, heteroarylalkyl or alkylheteroaryl comprises one or two coupling products; preferably at least one of said coupling products is triazolyl.

According to one embodiment, each L¹-R¹ is selected from —(CH₂)ₘ—R^{C}—(CH₂)ₙ—R¹, —O—(CH₂)ₘ—R^{C}—(CH₂)ₙ—R¹, —C(O)—(CH₂)ₘ—R^{C}—(CH₂)ₙ—R¹, —C(O)O—(CH₂)ₘ—R^{C}—(CH₂)ₙ—R¹, -phenyl-(CH₂)ₘ—R^{C}—(CH₂)ₙ—R¹ and -phenyl-O—(CH₂)ₘ—R^{C}—(CH₂)ₙ—R¹; wherein R^{C} is a coupling product, preferably triazolyl; m and n are independently an integer ranging from 1 to 8, preferably ranging from 1 to 4; and each R¹ is as defined hereinabove. According to one embodiment, each R¹ is 9-O-acetylated-sialic acid.

According to one embodiment, the glycocluster is a compound of formula (011), (005), (008) or (014)

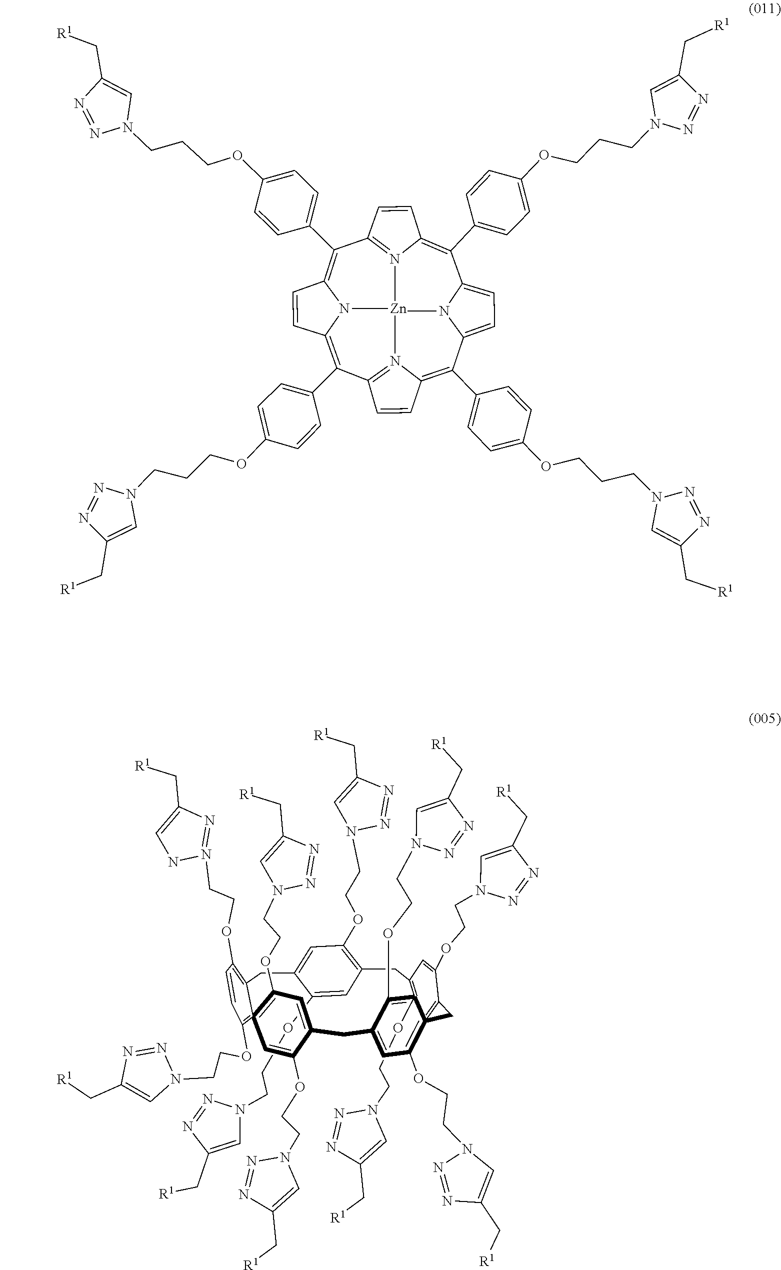

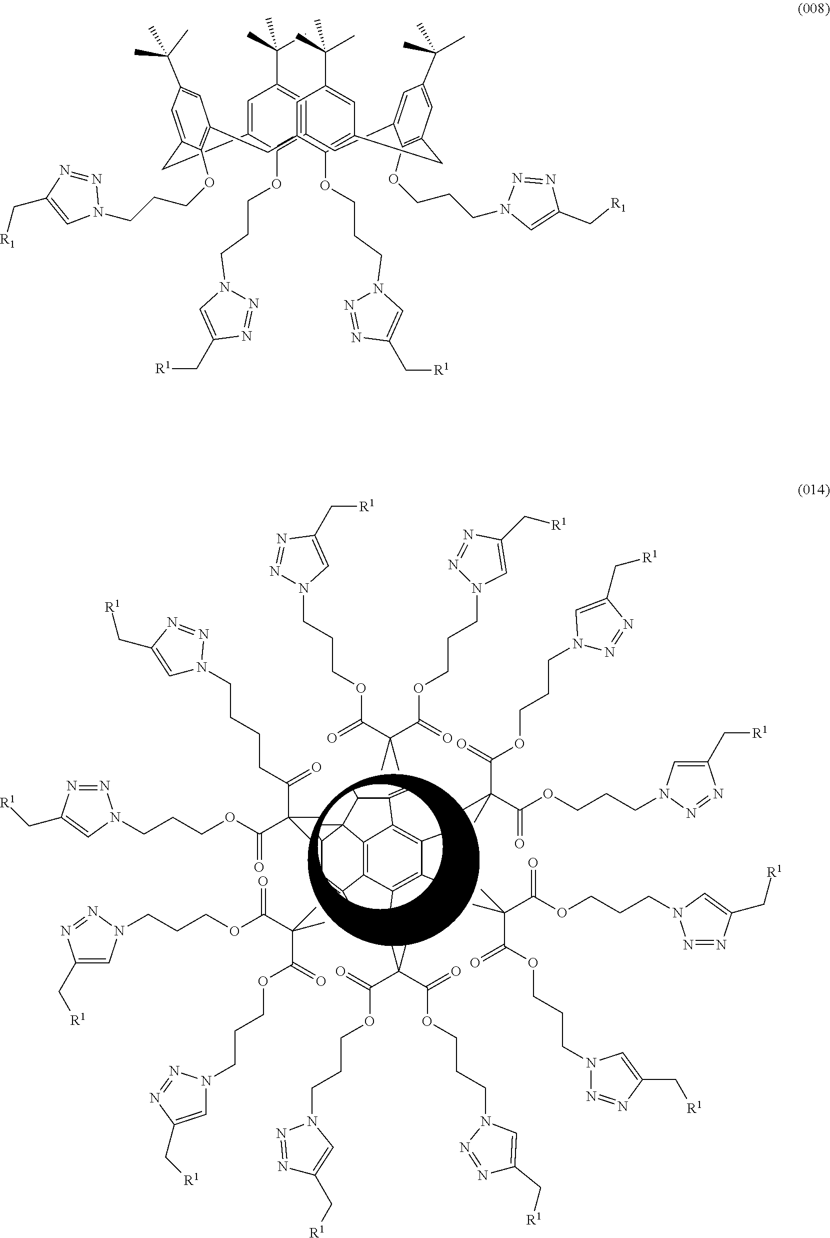

or a pharmaceutically acceptable salt or solvate thereof;
wherein each R¹ is of formula (9-AcSA)

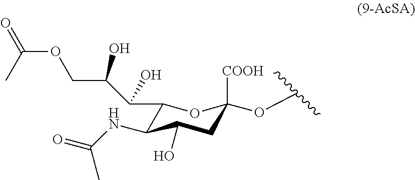

wherein the wavy line represents the point of attachment of R¹ to the glycocluster.

This invention also relates to a pharmaceutical composition comprising a glycocluster according to the invention and at least one pharmaceutically acceptable carrier. This invention also relates to a glycocluster according to the invention or a pharmaceutical composition according to the invention, for use as a medicament. This invention also relates to a glycocluster according to the invention or a pharmaceutical composition according to the invention, for use in the treatment and/or prevention of an infectious disease, preferably a coronavirus or picornavirus infection, more preferably a SARS-CoV-2 infection.

This invention also relates to a process for manufacturing a glycocluster according to the invention comprising a step of coupling of each of the acetylated-sialic acids with the macrocycle; preferably a coupling wherein the reaction between a terminal alkyne and an azide results in the formation of a triazolyl group.

### Definitions

In the present invention, the following terms have the following meanings:

### Chemical Definitions

When describing the compounds of the invention, the terms used are to be construed in accordance with the following definitions, unless indicated otherwise. Where chemical substituents are combinations of chemical groups, the point of attachment of the substituent to the molecule is by the last chemical group recited. For example, an arylalkyl substituent is bound to the rest of the molecule through the alkyl moiety and it may by represented as follows: “aryl-alkyl-”.

“Acetyl” or “ethanoyl”, represented by the symbol “Ac”, refers to the methyl acyl moiety of formula CH₃—C(O)—.

“Alkene” or “alkenyl” refer to a linear or branched hydrocarbon chain comprising at least one double bond and typically from 2 to 12 carbon atoms, preferably 3 to 6 carbon atoms. Non-limiting examples of alkenyl groups include ethenyl, 2-propenyl, 2-butenyl, 3-butenyl, 2-pentenyl and its isomers, 2-hexenyl and its isomers and 2,4-pentadienyl.

“Alkyl” refers to a saturated linear or branched hydrocarbon chain, typically comprising from 1 to 12 carbon atoms, preferably from 1 to 6 carbon atoms, more preferably from 1 to 3 carbon atoms. In the present invention, alkyl groups may be monovalent or divalent (i.e., “alkylene” groups are encompassed in “alkyl” definition). Non-limiting examples of alkyl groups include methyl, ethyl, n-propyl, i-propyl, n-butyl, i-butyl, s-butyl and t-butyl, pentyl and its isomers (e.g., n-pentyl, iso-pentyl), and hexyl and its isomers (e.g., n-hexyl, iso-hexyl). Preferred alkyl groups include methyl, ethyl, n-propyl, i-propyl, n-butyl, s-butyl and t-butyl.

“Alkylaryl” refers to an aryl group substituted by an alkyl group: alkyl-aryl-.

“Alkylheteroaryl” refers to a heteroaryl group substituted by an alkyl group: alkyl-heteroaryl-.

“Alkyne” or “alkynyl” refer to a linear or branched hydrocarbon chain comprising at least one triple bond and typically from 2 to 12 carbon atoms, preferably 3 to 6 carbon atoms. Examples of alkynyl groups include ethynyl, 2-propynyl, 2-butynyl, 3-butynyl, 2-pentynyl and its isomers, and 2-hexynyl and its isomers.

“Amide” refers to a functional group with the connectivity —(C═O)—NH—.

“Amido” refers to a —(C═O)—NH₂ group.

“Amine” refers to the —NH₂ group and to secondary amines —NHR wherein R is different from hydrogen, preferably wherein R is an alkyl group.

“Amino” refers to the group —NH₂.

“Aminooxy” refers to a —O—NH₂ group.

“Aryl” refers to a cyclic, polyunsaturated, aromatic hydrocarbyl group comprising at least one aromatic ring. Aryl groups may have a single ring (i.e., phenyl) or multiple aromatic rings fused together (e.g., naphthyl) or linked covalently. Typically, aryl groups have from 5 to 12 carbon atoms, preferably from 6 to 10 carbon atoms. The aromatic ring may optionally include one to two additional rings (either cycloalkyl, heterocycloalkyl or heteroaryl) fused thereto. Aryl is also intended to include the partially hydrogenated derivatives of the carbocyclic systems enumerated herein, as long as at least one ring is aromatic. Non-limiting examples of aryl groups include phenyl, biphenyl, biphenylenyl, 5- or 6-tetralinyl, naphthalen-1- or -2-yl, 4-, 5-, 6 or 7-indenyl, 1-2-, 3-, 4- or 5-acenaphthylenyl, 3-, 4- or 5-acenaphthenyl, 1- or 2-pentalenyl, 4- or 5-indanyl, 5-, 6-, 7- or 8-tetrahydronaphthyl, 1,2,3,4-tetrahydronaphthyl, 1,4-dihydronaphthyl, 1-, 2-, 3-, 4- or 5-pyrenyl. A preferred aryl group is phenyl.

“Arylalkyl” refers to an alkyl group substituted by an aryl group: aryl-alkyl-.

“Azide” refers to the group of formula —N₃.

“(Cₓ-C_{y})” preceding a group means that the group comprises from x to y carbon atoms, in accordance to common terminology in the chemistry field.

“Carboxylic acid” refers to the group of formula —COOH.

“Coupling function” refers to a functional group capable to react with another functional group to form a covalent linkage, such as a bond or a linear group of atoms. A coupling function which is reactive under suitable reaction conditions is thus capable of chemically reacting with another coupling function on a different molecule to form a new covalent linkage. A coupling function generally represents a point of attachment for another molecule. Coupling functions generally include nucleophiles, electrophiles and/or photoactivatable groups. Non-limiting examples of coupling functions include alcohol; alkene; alkyne (e.g., —C≡CH); amine; amide; aminooxy; anhydrides such as glutaric anhydride, succinic anhydride or maleic anhydride; azide; carboxylic acid; activated carboxylic acid such as acid anhydride or acid halide; chloroformate; activated ester such as N-hydroxysuccinimide ester, N-hydroxyglutarimide ester or maleimide ester; glutamate; halide (halogen atom); haloacetamide (i.e., —NH—C(O)CH₂X moiety wherein X is a halogen atom) such as chloroacetamide, bromoacetamide or iodoacetamide; hydrazide; isocyanate; isothiocyanate; ketone; maleimide; norbornen; phosphonic acid; siloxy; tetrazine and thiol. The reaction between two coupling functions can result in a “coupling product” as defined herein.

“Coupling product” refers to a residue of a coupling function that results from the reaction between two coupling functions in different molecules, for example a functionally related group of atoms (such as amide —C(O)—NH— group or a double bond) or a heterocycle (such as a divalent triazolyl group). In other words, a coupling product is what remains of one or two coupling function(s) after the coupling reaction between the two coupling functions. For example, the coupling reaction between two coupling functions A and B can lead to the following coupling products as shown on Table 1 below, wherein X represents a halogen atom (e.g., Br or Cl). A coupling product may be comprised in a “linker” as defined herein.

TABLE 1
Examples of coupling functions and products

| R-coupling function A | R′-coupling function B | R-coupling product-R′ |
|---|---|---|
| R—N₃ | R′—C≡CH | R-triazolyl-R′ |
| R—NH₂ | R′—COOH | R—NH(C═O)—R′ |
| R—SH | R′—SH | R—S—S—R′ |
| R—OH | R′-(epoxide) | R—OCH₂CH(OH)—R′ |
| R—NH₂ | R′-(epoxide) | R—NHCH₂CH(OH)—R′ |
| R—SH | R′-(epoxide) | R—SCH₂CH(OH)—R′ |
| R—NH₂ | R′—CHO | R—N═CH—R′ |
| R—NH₂ | R′—NCO | R—NH(C═O)NH—R |
| R—NH₂ | R′—NCS | R—NH(C═S)NH—R′ |
| R—SH | R′—(C═O)CH₃ | R—(C═O)CH₂S—R′ |
| R—SH | R′—O(C═O)X | R—S(C═O)O—R′ |
| R—CH═CH₂ | R′—SH | R—CH₂CH₂S—R′ |
| R—OH | R′—NCO | R—O(C═O)NH—R′ |
| R—SH | R′—(C═O)CH₂X | R—SCH₂(C═O)—R′ |
| R—NH₂ | R′—(C═O)N₃ | R—NH(C═O)—R′ |
| R—COOH | R′—COOH | R—(C═O)O(C═O)—R′ |
| R—SH | R′—X (R-halogen) | R—S—R′ |
| R—NH₂ | R′—CH₂—C(NH₂⁺)—OCH₃ | R—NH—C(NH₂⁺)—CH₂—R′ |
| | (“Pinner salt”) | |
| R—NHNH₂ | R′—CHO | R—NHNH═CH—R′ |
| R—CH═CH—B(OH)₂ | R′—X | R—CH═CH—R′ |
| R—C≡CH | R′—X | R—C≡C—R′ |

“Covalently linked” means that two moieties are covalently bound together either directly, i.e., by means of a single, double or triple covalent bond (typically a single bond), or indirectly, i.e., by means of a “linker” as described herein, which comprises a plurality of covalent bonds.

“Glycocluster” refers to a cluster of glycans, i.e., a molecule or ensemble of molecules comprising a plurality of glycan units. Thus, a glycocluster comprises at least two polysaccharide, oligosaccharide and/or monosaccharide moieties, typically at least two monosaccharides. In a glycocluster, the glycan units are grouped by means of their bonding to a common scaffold (e.g., a macrocycle, a polymer or a metal nanoparticle) and are relatively close to each other. Glycoclusters are often used for drug delivery, however in the present invention they may be used as inhibitors of cell binding and/or infectivity for use in the treatment of infectious diseases.

“Halide”, “halo” or “halogen” refers to a fluorine, chlorine, bromine or iodine atom; preferably a chlorine or bromine atom.

“Heteroalkyl” refers to an alkyl group as defined hereinabove wherein one or more carbon atoms are replaced by a heteroatom selected from oxygen, nitrogen and sulfur. In heteroalkyl groups, the heteroatoms are bound along the alkyl chain only to carbon atoms, i.e., each heteroatom is separated from any other heteroatom by at least one carbon atom. The nitrogen and sulfur heteroatoms may optionally be oxidized and the nitrogen heteroatoms may optionally be quaternized. A heteroalkyl is bond to another group or molecule only through a carbon atom, i.e., the bounding atom is not selected among the heteroatoms included in the heteroalkyl group. Non-limiting examples of heteroalkyl include alkoxy, ethers and polyethers, secondary amines, tertiary amines and thioethers.

“Heteroaryl” refers to aromatic rings or aromatic ring systems comprising from 5 to 12 carbon atoms, preferably from 6 to 10 carbon atoms, having one or two rings which are fused together or linked covalently, wherein at least one ring is aromatic, and wherein one or more carbon atoms in one or more of these rings is replaced by oxygen, nitrogen and/or sulfur atoms. “Heteroaryl” may also be viewed as an “aryl” group as defined herein, wherein at least one carbon atom in the aryl group is replaced with a heteroatom and wherein the resulting molecule is chemically stable. The nitrogen and sulfur heteroatoms may optionally be oxidized and the nitrogen heteroatoms may optionally be quaternized. Non-limiting examples of heteroaryl groups include furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, thienopyridinyl, purinyl, imidazo[1,2-a]pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl and quinoxalinyl.

“Heteroarylalkyl” refers to an alkyl group substituted by a heteroaryl group: heteroaryl-alkyl-.

“Hydroxyl” refers to —OH group.

“Ketone” refers to a functional group with the connectivity C—(C═O)—C.

“Linker” refers to a moiety that covalently binds two molecules to one another and comprises a series of multivalent atoms selected from C, N, O, S and P bound together by stable covalent bonds. The moiety typically incorporates 1 to 30 atoms, for example 1, 2, 3, 4, 5, 6, 7, 8, 9, 10, 15, 20, 25 or 30. A linker may be linear or non-linear, some linkers have pendant side chains or pendant functional groups or both. In one embodiment, a linker is composed of any combination of single, double, triple or aromatic carbon-carbon bonds, carbon-nitrogen bonds, nitrogen-nitrogen bonds, carbon-oxygen bonds and carbon-sulfur bonds. In one embodiment, a linker consists of a combination of moieties selected from alkyl, —C(O)NH—, —C(O)O—, —NH—, —S—, —O—, —C(O)—, —S(O)—, —S(O)₂ and 5- or 6-membered monocyclic aryls or heteroaryls. In one embodiment, the linker comprises at least one “coupling product” as defined herein, typically one or two coupling products. In this context, “comprise” means that the linker can be interrupted by at least one coupling product (i.e., the coupling product is incorporated into the atomic chain of the linker) and/or that the linker can end with at least one coupling product (i.e., the coupling product terminates the linker). In this embodiment, the coupling product is considered part of the linker, which means in particular that the atoms of the coupling product are counted among the total number of atoms of the linker.

“Macrocycle” refers to a molecule or ion containing a 12- or more membered ring such as pillararenes, calixarenes, porphyrins, fullerenes, crown ethers and cyclodextrins. For the sake of simplicity and clarity, in the present application “macrocycle” term and any specific genus thereof (e.g., pillararenes, calixarenes, porphyrins and fullerenes) refer both to the macrocycle per se and to any macrocycle-based moiety resulting from the coupling of a macrocycle with another molecule, either directly by a single bound or through a “linker” as defined herein. If necessary, the latter will be specifically referred to as a “residue of macrocycle” or “macrocycle residue”.

“Peptide” refers to a linear polymer of amino acids of less than 50 amino acids linked together by peptide bonds.

“Sialic acid” or “SA” refers to a monosaccharide belonging to a class of alpha-keto acid sugars with a nine-carbon backbone, in accordance with the general knowledge in the art. In particular, sialic acid refers to acetylneuraminic acid (Neu5Ac) of the following formulae.

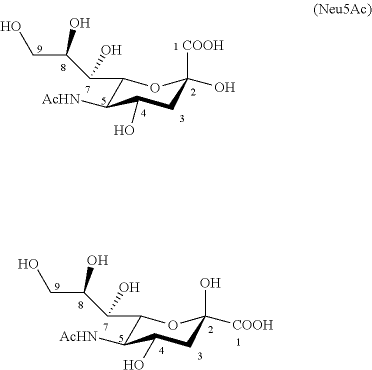

The configuration wherein the carboxylic acid is in the axial position is the alpha-anomer, whereas the configuration wherein the carboxylic acid is in the equatorial position is the beta-anomer. When bound to another molecule, sialic acid is mainly the alpha-anomer. Carbon numbering for sialic acids is as shown in the above formulae (left: alpha-anomer, right: beta-anomer).

Sialic acids may be “acetylated” in accordance with the general meaning in the art, i.e., a group in the sialic acid (typically a hydrogen atom) may be replaced by an acetyl group. Although Neu5Ac includes an acetylated amine (AcHN), in the present application, only sialic acids wherein the acetylation is on both a hydroxyl (OH) group and the amine are considered “acetylated” in the sense of the invention. In particular, an “acetylated-sialic acid” is a sialic acid wherein at least one hydroxyl (—OH) on positions 4, 7, 8 and 9 has been substituted by an acetyl group, thereby resulting in an acetate moiety (—OC(O)CH₃). Synthetic methods for the acetylation of one or more positions in a sialic acid, including esterification reactions with acetic acid, are well-known in the art. In particular, an acetylated-sialic acid may refer to 9-O-acetyl-sialic acid (9-AcSA) of the following formulae (left: alpha-anomer, right: beta-anomer).

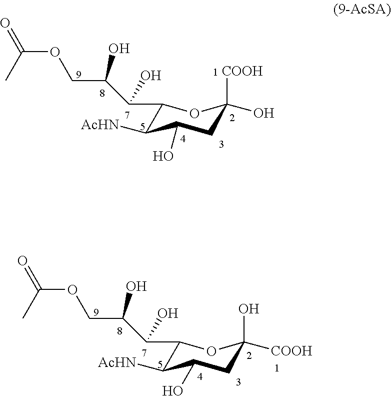

A sialic acid or acetylated-sialic acid may be bound to another compound, be they natural or artificial, either directly by a single covalent bond or through a linker. For example, in the invention, sialic acids and/or acetylated-sialic acids are bound to a macrocycle in order to form a glycocluster. Non-acetylated hydroxyls may in particular be used as coupling functions to obtain a covalent linkage. For the sake of simplicity and clarity, in the present application “sialic acid” term and any specific genus thereof (e.g., acetylated-sialic acid) refer both to the sialic acid monosaccharide per se and to any sialic acid-based moiety resulting from the coupling of sialic acid with another molecule, either directly by a single bound or through a “linker” as defined herein. If necessary, the latter will be specifically referred to as a “residue of sialic acid” or “sialic acid residue”. When a sialic acid is bound to another molecule by means of a non-acetylated hydroxyl (e.g., the hydroxyl on position 2), the remaining oxygen is considered as part of the sialic acid residue for definition and representation purposes.

“Siloxy” refers to the function —O—Si(R)₃ wherein R represents for example alkyl or aryl.

“Triazolyl” refers to a monovalent, divalent or trivalent derivative of the heteroaryl of general formula

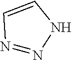

(i.e., triazole), such as for example

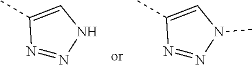

groups.

### General Definitions

“About” is used herein to mean approximately, roughly, around, or in the region of. The term “about” preceding a figure means plus or less 10% of the value of the figure. When the term “about” is used in conjunction with a numerical range, it modifies that range by extending the boundaries above and below the numerical values set forth by 10%.

“Administration”, or a variant thereof (e.g., “administering”), means providing a therapeutic agent (e.g., a compound of the invention) alone or as part of a pharmaceutically acceptable composition, to the patient in whom/which the condition, symptom, or disease is to be treated and/or prevented.

“Human” refers to a male or female subject at any stage of development, including neonate, infant, juvenile, adolescent and adult.

“Patient” refers to an animal, typically a warm-blooded animal, preferably a human, who/which is awaiting the receipt of, or is receiving medical care, or is/will be the object of a medical procedure. A patient may also be the subject of preventive care or procedure.

“Pharmaceutically acceptable” means that the ingredients of a composition are compatible with each other and not deleterious to the patient to which/whom it is administered.

“Pharmaceutically acceptable carrier” refers to an excipient that does not produce an adverse, allergic or other untoward reaction when administered to an animal, preferably a human. It includes any and all solvents, dispersion media, coatings, antibacterial and antifungal agents, isotonic and absorption delaying agents and the like. For human administration, preparations should meet sterility, pyrogenicity, general safety and purity standards as required by regulatory offices, such as, e.g., FDA Office or EMA.

“Prevent”, “preventing” and “prevention” refer to delaying or precluding the onset of a condition and/or disease and/or any one of its attendant symptoms, barring a patient from acquiring a condition or disease, or reducing the risk for a patient of acquiring a condition and/or disease and/or any one of its attendant symptoms.

“Prodrug” refers to a pharmacologically acceptable derivative of a therapeutic agent (e.g., a compound of the invention) whose in vivo biotransformation product is the therapeutic agent (active drug). Prodrugs are typically characterized by increased bioavailability and are readily metabolized in vivo into the active compounds. Non-limiting examples of prodrugs include amide prodrugs and carboxylic acid ester prodrugs, in particular alkyl esters, cycloalkyl esters and aryl esters.

“Solvate” refers to molecular complex comprising a compound along with stoichiometric or sub-stoichiometric amounts of one or more molecules of one or more solvents, typically the solvent is a pharmaceutically acceptable solvent such as, for example, ethanol. The term “hydrate” refers to when the solvent is water (H₂O).

“Therapeutic agent”, “active pharmaceutical ingredient” and “active ingredient” refer to a compound for therapeutic use and relating to health. Especially, a therapeutic agent (e.g., a compound of the invention) may be indicated for treating and/or preventing a disease, preferably an infectious disease. An active ingredient may also be indicated for improving the therapeutic activity of another therapeutic agent.

“Therapeutically effective amount” (in short “effective amount”) refers to the amount of a therapeutic agent (e.g., a compound of the invention) that is sufficient to achieve the desired therapeutic or prophylactic effect in the patient to which/whom it is administered.

“Treat”, “treating” and “treatment” refer to alleviating, attenuating or abrogating a condition and/or disease and/or any one of its attendant symptoms, e.g., an infectious disease.

### DETAILED DESCRIPTION

### Glycocluster

This invention relates to a glycocluster comprising at least two acetylated-sialic acids covalently linked to a macrocycle. In the present application, “the glycocluster” and “the compound of the invention” and similar wordings are synonyms.

According to one embodiment, the glycocluster comprises at least 3, 4, 5, 6, 7, 8, 9, 10, 11, 12, 14, 15 or 16 acetylated sialic acids covalently linked to the macrocycle. In one embodiment, the glycocluster comprises at least four acetylated sialic acids covalently linked to the macrocycle. According to one embodiment, the glycocluster comprises four acetylated sialic acids covalently linked to the macrocycle (“tetramer”). According to one embodiment, the glycocluster comprises ten acetylated sialic acids covalently linked to the macrocycle (“decamer”). According to one embodiment, the glycocluster comprises twelve acetylated sialic acids covalently linked to the macrocycle (“dodecamer”).

According to one embodiment, each acetylated-sialic acid not fully acetylated, i.e., wherein at least one OH group on positions 4, 7, 8 and 9 is not substituted by an acetyl. In one embodiment, each acetylated-sialic acid is acetylated at 1, 2, 3 or 4 positions among positions 4, 7, 8 and 9. In one embodiment, each acetylated-sialic acid is acetylated at 1, 2 or 3 positions. In one embodiment, each acetylated-sialic acid is acetylated at 1 or 2 positions. In one embodiment, each acetylated-sialic acid is acetylated at positions 7 and 9.

According to one embodiment, each acetylated-sialic acid comprises at least one OH group. In one embodiment, each acetylated-sialic acid comprises one OH group. In one embodiment, each acetylated-sialic acid comprises two OH groups. In one embodiment, each acetylated-sialic acid comprises three OH groups. In one embodiment, each acetylated-sialic acid comprises four OH groups.

According to one embodiment, each acetylated-sialic acid is acetylated at one position only, i.e., each acetylated-sialic acid is independently selected from, 4-O-acetylated-sialic acid, 7-O-acetylated-sialic acid, 8-O-acetylated-sialic acid and 9-O-acetylated-sialic acid. According to one embodiment, each acetylated-sialic acid is independently selected from 7-O-acetylated-sialic acid, 8-O-acetylated-sialic acid and 9-O-acetylated-sialic acid. According to one embodiment, each acetylated-sialic acid is independently selected from 7-O-acetylated-sialic acid and 9-O-acetylated-sialic acid. In one embodiment, each acetylated-sialic acid is 4-O-acetylated-sialic acid. In one embodiment, each acetylated-sialic acid is 7-O-acetylated-sialic acid. In one embodiment, each acetylated-sialic acid is 8-O-acetylated-sialic acid. In one embodiment, each acetylated-sialic acid is 9-O-acetylated-sialic acid.

Without being bound by any theory, the Applicants believe that sialic acids that are not fully acetylated, in particular sialic acids wherein only one OH is acetylated, are advantageous in terms of virus affinity or inhibition, in particular over SARS-CoV-2.

Typically, the macrocycle is selected from porphyrins, pillararenes, calixarenes and fullerenes.

According to one embodiment, the macrocycle is selected from porphyrins. In one embodiment, the porphyrin is selected from porphin or tetraphenylporphyrin. In one embodiment, the porphyrin has a metal cation coordinated by the four nitrogen atoms and no hydrogen attached to the nitrogen atoms (“porphyrin chelate”). Non-limiting examples include [Zn(porphin)] and [Zn(tetraphenylporphyrin)]. In another embodiment, the porphyrin does not have any cation coordinated by the four nitrogen atoms and two hydrogens are attached to the nitrogen atoms (“porphyrin free base”).

According to one embodiment, the macrocycle is selected from pillararenes. In one embodiment, the pillararene is selected from pillar[5]arenes.

According to one embodiment, the macrocycle is selected from calixarenes. In one embodiment, the calixarene is selected from calix[4]arenes.

According to one embodiment, the macrocycle is selected from fullerenes. In one embodiment, the macrocycle is selected from C₆₀-fullerenes, also called buckminsterfullerenes.

In the invention, the macrocycle may be non-substituted (unsubstituted) apart from the acetylated-sialic acid substituents. In the invention, the macrocycle may comprise further substituents, e.g., water-solubilizing substituents or substituents susceptible to interact with a virus.

According to one embodiment, the glycocluster comprises at least one angiotensin-converting enzyme 2 (ACE2) binding inhibitor. In one embodiment, the ACE2 binding inhibitor is an ACE2 binding inhibitor peptide or protein. In one embodiment, the ACE2 binding inhibitor is an ACE2 binding inhibitor peptide. In one embodiment, the ACE2 binding inhibitor is an ACE2 binding inhibitor protein. In one embodiment, the ACE2 binding inhibitor is an anti-ACE2 antibody or antigen-binding fragments thereof, such as an anti-ACE2 monoclonal antibody. In one embodiment, the ACE2 binding inhibitor peptide is designed according to the sequence of the ACE2 receptor in complex with the RBD domain of the S1 glycoprotein. In one embodiment, the ACE2 binding inhibitor peptide is selected from the peptides [22-44] consisting of the amino acid sequence EEQAKTFLDKFNHEAEDLFYQSS (SEQ ID NO: 1), [351-357] consisting of the amino acid sequence LGKGDFR (SEQ ID NO: 2), [22-57] consisting of the amino acid sequence EEQAKTFLDKFNHEAEDLFYQSSLASWNYNTNITEE (SEQ ID NO: 3) and [22-44-g-351-357] consisting of the amino acid sequence EEQAKTFLDKFNHEAEDLFYQSSGLGKGDFR (SEQ ID NO: 4); as described in reference document: Yang, J. et al., Nature Communications, 2020, Vol. 11, Article number: 4541.

According to one embodiment, the glycocluster comprises at least one other substituent susceptible to bind with S protein such as antibody, antibody fragments, nanobody or lectin.

According to one embodiment, the glycocluster is a compound of formula (I)

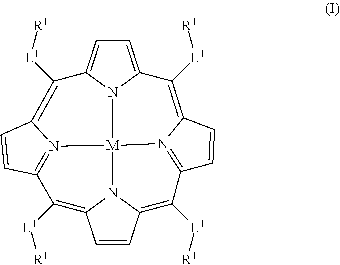

In formula (I), M is a metal cation such as zinc, iron, copper, manganese, silver, gold, cobalt, nickel, tin, cadmium, lead or vanadium cations. In one embodiment, the metal cation has a positive charge of two or three, preferably a positive charge of two. In one embodiment, M is a metal cation selected from zinc (II), iron (II), iron (III), copper (II), copper (III), manganese (II), manganese (III), silver (II), silver (III), gold (III), cobalt (II), cobalt (III), nickel (II), nickel (III), tin (II), cadmium (II), lead (II), vanadium (II) and vanadium (III).

According to one embodiment, the glycocluster is a compound of formula (Ia)

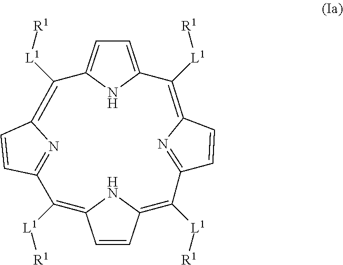

In formulae (I) and (Ia), each L¹ is independently a single bond or a linker selected from alkyl, heteroalkyl, alkenyl, alkynyl, aryl, alkylaryl, arylalkyl, heteroaryl, heteroarylalkyl and alkylheteroaryl; wherein the alkyl, heteroalkyl, alkenyl, alkynyl, aryl, alkylaryl, arylalkyl, heteroaryl, heteroarylalkyl or alkylheteroaryl optionally comprises at least one coupling product. In one embodiment, each L¹ is a linker selected from alkyl, heteroalkyl, alkylaryl, arylalkyl, heteroarylalkyl and alkylheteroaryl; wherein the alkyl, heteroalkyl, alkylaryl, arylalkyl, heteroarylalkyl or alkylheteroaryl comprises at least one coupling product. In one embodiment, the linker comprises one or two coupling product(s). In one embodiment, the linker comprises one coupling product (i.e., only one coupling product). In one embodiment, at least one of the coupling products is triazolyl.

In formulae (I) and (Ia), each R¹ is independently selected from acetylated-sialic acids and ACE2 binding inhibitors; provided that at least two R¹ are acetylated-sialic acids.

In one embodiment, each L¹-R¹ is selected from —(CH₂)ₘ—R^{C}—(CH₂)ₙ—R¹, —O—(CH₂)ₘ—R^{C}—(CH₂)ₙ—R¹, —C(O)—(CH₂)ₘ—R^{C}—(CH₂)ₙ—R¹, —C(O)O—(CH₂)ₘ—R^{C}—(CH₂)ₙ—R¹, -phenyl-(CH₂)ₘ—R^{C}—(CH₂)ₙ—R¹ and -phenyl-O—(CH₂)ₘ—R^{C}—(CH₂)ₙ—R¹; wherein R^{C} is a coupling product, and m and n are independently an integer ranging from 1 to 8. In one embodiment, R^{C} is triazolyl. In one embodiment, m is an integer ranging from 1 to 6, preferably ranging from 1 to 4, more preferably ranging from 2 to 4, more preferably 2 or 3. In one embodiment, n is an integer ranging from 1 to 4, preferably ranging from 1 to 3, more preferably 1 or 2, more preferably 1.

In one embodiment, each L¹-R¹ is -phenyl-O—(CH₂)ₘ—R^{C}—(CH₂)ₙ—R¹; wherein R^{C} is a coupling product, and m and n are independently an integer ranging from 1 to 8.

In one embodiment, each R¹ is selected from 7-O-acetylated-sialic acid and 9-O-acetylated-sialic acid. In one embodiment, each R¹ is 7-O-acetylated-sialic acid. In one embodiment, each R¹ is 9-O-acetylated-sialic acid.

According to one embodiment, the glycocluster is a compound of formula (II)

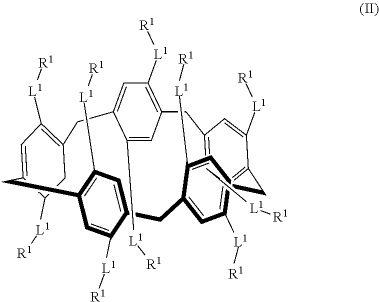

wherein L¹ and R¹ are as defined hereinabove under formulae (I) and (Ia).

In one embodiment, each L¹-R¹ is —O—(CH₂)ₘ—R^{C}—(CH₂)ₙ—R¹; wherein R^{C} is a coupling product, and m and n are independently an integer ranging from 1 to 8. In one embodiment, R^{C} is triazolyl. In one embodiment, m is an integer ranging from 1 to 6, preferably ranging from 1 to 4, more preferably ranging from 2 to 4, more preferably 2 or 3. In one embodiment, n is an integer ranging from 1 to 4, preferably ranging from 1 to 3, more preferably 1 or 2, more preferably 1.

According to one embodiment, the glycocluster is a compound of formula (III)

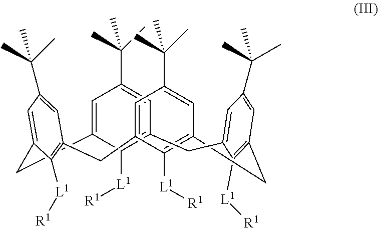

wherein L¹ and R¹ are as defined hereinabove under formulae (I) and (Ia).

In one embodiment, each L¹-R¹ is —O—(CH₂)ₘ—R^{C}—(CH₂)ₙ—R¹; wherein R^{C} is a coupling product, and m and n are independently an integer ranging from 1 to 8. In one embodiment, R^{C} is triazolyl. In one embodiment, m is an integer ranging from 1 to 6, preferably ranging from 1 to 4, more preferably ranging from 2 to 4, more preferably 2 or 3. In one embodiment, n is an integer ranging from 1 to 4, preferably ranging from 1 to 3, more preferably 1 or 2, more preferably 1.

According to one embodiment, the glycocluster is a compound of formula (IV)

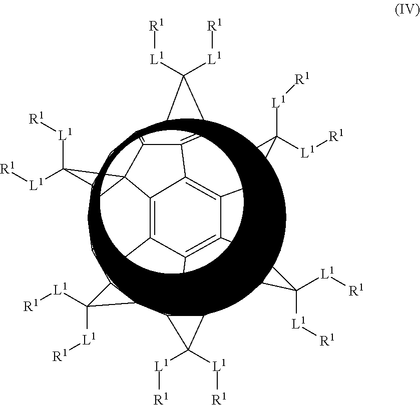

wherein L¹ and R¹ are as defined hereinabove under formulae (I) and (Ia).

In one embodiment, each L¹-R¹ is —C(O)O—(CH₂)ₘ—R^{C}—(CH₂)ₙ—R¹; wherein R^{C} is a coupling product, and m and n are independently an integer ranging from 1 to 8. In one embodiment, R^{C} is triazolyl. In one embodiment, m is an integer ranging from 1 to 6, preferably ranging from 1 to 4, more preferably ranging from 2 to 4, more preferably 2 or 3. In one embodiment, n is an integer ranging from 1 to 4, preferably ranging from 1 to 3, more preferably 1 or 2, more preferably 1.

According to one embodiment, the glycocluster is a compound of formula (011)

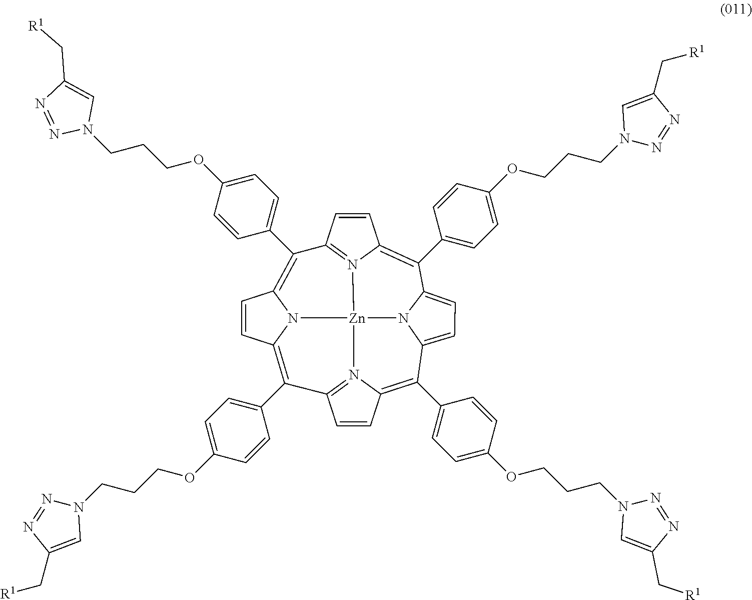

or a pharmaceutically acceptable salt or solvate thereof.

According to one embodiment, the glycocluster is a compound of formula (005)

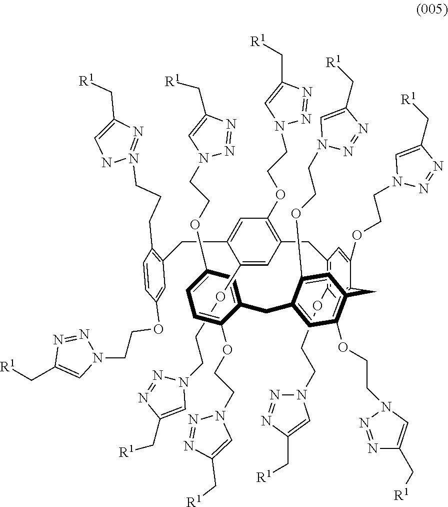

or a pharmaceutically acceptable salt or solvate thereof.

According to one embodiment, the glycocluster is a compound of formula (008)

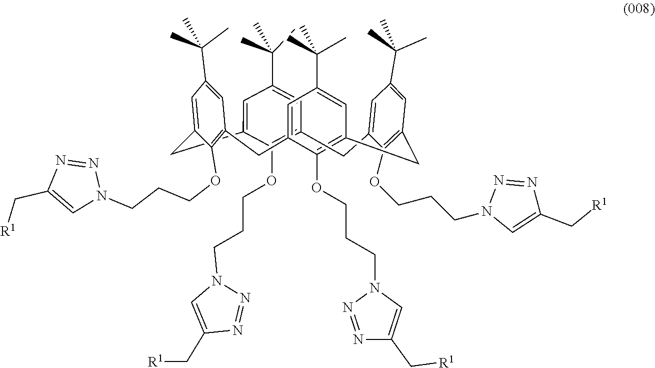

or a pharmaceutically acceptable salt or solvate thereof.

According to one embodiment, the glycocluster is a compound of formula (014)

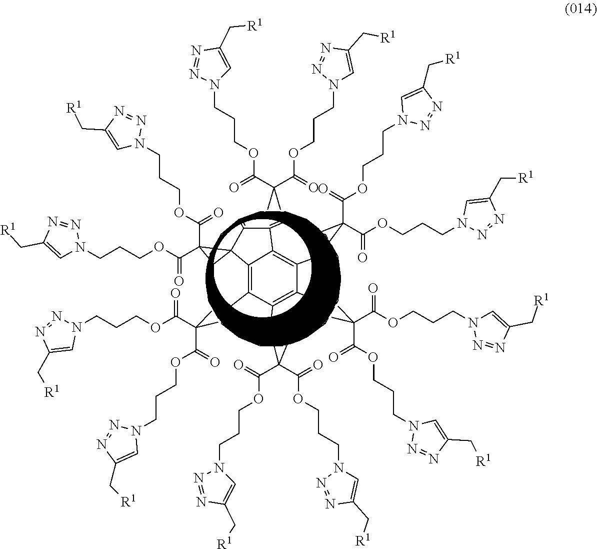

or a pharmaceutically acceptable salt or solvate thereof.

In formulae (011), (005), (008) and (014) hereinabove, each R¹ is as described under formulae (I) and (Ia).

In one embodiment, in formulae (011), (005), (008) and (014) hereinabove, each R¹ is of formula (9AcSA)

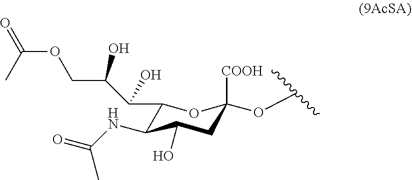

wherein the wavy line represents the point of attachment of R¹ to the glycocluster.

In one embodiment, the glycocluster is selected from the compound of formula (011), (005), (008) and (014) hereinabove, wherein each R¹ is of formula (9AcSA), or a pharmaceutically acceptable salt or solvate thereof, i.e., compounds 011, 005, 008 and 014 as represented in Example 1 herein, or a pharmaceutically acceptable salt or solvate thereof.

All references herein to a compound of the invention (e.g., “glycocluster” or “formula (I)”) include references to salts, preferably pharmaceutically acceptable salts, solvates, multi component complexes and/or liquid crystals thereof. All references herein to a compound of the invention include references to polymorphs and/or crystal habits thereof. All references to a compound of the invention include references to pharmaceutically acceptable prodrugs thereof. All references to a compound of the invention include references to isotopically-labelled compounds, including deuterated compounds.

A compound of the invention (e.g., “glycocluster” or “formula (I)”) and subformulae thereof contains at least one asymmetric centre(s) and thus may exist as different stereoisomeric forms. Accordingly, all references to a compound of the invention include references to all possible stereoisomers and includes not only the racemic compounds but the individual enantiomers and their non-racemic mixtures as well. When a compound is desired as a single enantiomer, such single enantiomer may be obtained by stereospecific synthesis, by resolution of the final product or any convenient intermediate, or by chiral chromatographic methods as each are known in the art. Resolution of the final product, an intermediate, or a starting material may be carried out by any suitable method known in the art.

The compounds of the invention may be in the form of pharmaceutically acceptable salts. Pharmaceutically acceptable salts include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinafoate salts. Suitable base salts are formed from bases which form non-toxic salts. Examples include the aluminium, arginine, benzathine, calcium, choline, diethylamine, 2-(diethylamino)ethanol, diolamine, ethanolamine, glycine, 4-(2-hydroxyethyl)-morpholine, lysine, magnesium, meglumine, morpholine, olamine, potassium, sodium, tromethamine and zinc salts. Hemisalts of acids and bases may also be formed, for example, hemisulphate and hemicalcium salts. When a compound contains an acidic group as well as a basic group the compound may also form internal salts, and such compounds are within the scope of the invention. When a compound contains a hydrogen-donating heteroatom (e.g., NH), the invention also covers salts and/or isomers formed by transfer of said hydrogen atom to a basic group or atom within the molecule.

Pharmaceutically acceptable salts of compounds of the invention may be prepared by one or more of these methods: (i) by reacting the compound with the desired acid; (ii) by reacting the compound with the desired base; (iii) by removing an acid- or base-labile protecting group from a suitable precursor of the compound or by ring-opening a suitable cyclic precursor, e.g., a lactone or lactam, using the desired acid; and/or (iv) by converting one salt of the compound to another by reaction with an appropriate acid or by means of a suitable ion exchange column. All these reactions are typically carried out in solution. The salt may precipitate from solution and be collected by filtration or may be recovered by evaporation of the solvent. The degree of ionization in the salt may vary from completely ionized to almost non-ionized.

### Pharmaceutical Composition

This invention also relates to a pharmaceutical composition comprising a compound of the invention as described herein and at least one pharmaceutically acceptable carrier.

According to one embodiment, the pharmaceutical composition further comprises at least another therapeutic agent. In one embodiment, the therapeutic agent is an antiviral agent. In one embodiment, the at least another therapeutic agent is an angiotensin-converting enzyme 2 (ACE2) binding inhibitor, as described herein. In one particular embodiment, the enzyme 2 (ACE2) binding inhibitor is vectorized by the glycocluster.

The compound of the invention may be formulated, alone or together, in suitable dosage unit formulations containing conventional non-toxic pharmaceutically acceptable carriers, adjuvants and vehicles appropriate for each route of administration.

### Medical Use and Methods of Treatment

This invention also relates to a compound of the invention as described herein, or a pharmaceutical composition of the invention as described herein, for use as a medicament.

This invention also relates to a compound of the invention as described herein, or a pharmaceutical composition of the invention as described herein, for use in the treatment and/or prevention of an infectious disease.

According to one embodiment, the infectious disease is a coronavirus infection or a picornavirus infection. In one embodiment, the infectious disease is a coronavirus infection.

In one embodiment, the coronavirus is an alpha coronavirus or a beta coronavirus, preferably a beta coronavirus. Non-limiting examples of alpha coronaviruses include human coronavirus 229E (HCoV-229E) and human coronavirus NL63 (HcoV-NL63) also sometimes known as HcoV-NH or New Haven human coronavirus. Non-limiting examples of beta coronaviruses include human coronavirus OC43 (HcoV-OC43), human coronavirus HKU1 (HcoV-HKU1), Middle East respiratory syndrome-related coronavirus (MERS-CoV) previously known as novel coronavirus 2012 or HcoV-EMC, severe acute respiratory syndrome coronavirus (SARS-CoV) also known as SARS-CoV-1 or SARS-classic, and severe acute respiratory syndrome coronavirus (SARS-CoV-2) also known as 2019-nCoV or novel coronavirus 2019. In one embodiment, the coronavirus is selected from HcoV-229E, HcoV-NL63, HcoV-OC43, HcoV-HKU1, MERS-CoV, SARS-CoV-1 and SARS-CoV-2. In one embodiment, the coronavirus is selected from MERS-CoV, SARS-CoV-1 and SARS-CoV-2.

According to one embodiment, the coronavirus is a SARS coronavirus. In one embodiment, the coronavirus is SARS-CoV-1 or SARS-CoV-2. In one embodiment, the coronavirus is SARS-CoV (also referred to as SARS-CoV-1) causing severe acute respiratory syndrome (SARS). In one embodiment, the coronavirus is SARS-CoV-2 causing COVID-19.

In the present application, any reference to a “coronavirus” or to “SARS-CoV-2” encompass any variant thereof currently identified. According to one embodiment, the coronavirus is an original haplotype of the SARS-CoV-2 pandemic (lineage A or B), or a variant thereof. In one embodiment, the coronavirus is SARS-CoV-2 Alpha (lineage B.1.1.7, and sub-lineages thereof). In one embodiment, the coronavirus is SARS-CoV-2 Beta (lineage B.1.351, and sub-lineages thereof). In one embodiment, the coronavirus is SARS-CoV-2 Gamma (lineage P.1, and sub-lineages thereof). In one embodiment, the coronavirus is SARS-CoV-2 Delta (lineages B.1.617.2, XD, XF, XS, and sub-lineages thereof). In one embodiment, the coronavirus is SARS-CoV-2 Epsilon (lineages B.1.427, B1.429, and sub-lineages thereof). In one embodiment, the coronavirus is SARS-CoV-2 Zeta (lineage P.2, and sub-lineages thereof). In one embodiment, the coronavirus is SARS-CoV-2 Eta (lineage B.1.525, and sub-lineages thereof). In one embodiment, the coronavirus is SARS-CoV-2 Theta (lineage P.3, and sub-lineages thereof). In one embodiment, the coronavirus is SARS-CoV-2 Iota (lineage B.1.526, and sub-lineages thereof). In one embodiment, the coronavirus is SARS-CoV-2 Kappa (lineage B.1.617.1, and sub-lineages thereof). In one embodiment, the coronavirus is SARS-CoV-2 Lambda (lineage C.37, and sub-lineages thereof). In one embodiment, the coronavirus is SARS-CoV-2 Mu (lineage B.1.621, and sub-lineages thereof). In one embodiment, the coronavirus is SARS-CoV-2 Omicron (lineages B.1.1.529, BA.1, BA.2, BA.3, BA.4, BA.5, XE, and sub-lineages thereof). A list of all SARS-CoV-2 variants can be found on the cov-lineages website, at https://cov-lineages.org/lineage list.html.

In one embodiment, the COVID-19 is moderate COVID-19. In one embodiment, the COVID-19 is mild-to-moderate COVID-19. In one embodiment, the COVID-19 is mild COVID-19. In one embodiment, the COVID-19 is mild-to-severe COVID-19. In one embodiment, the COVID-19 is severe COVID-19. The subject suffering from COVID-19 may or may not be hospitalized.

In one embodiment, COVID-19 severity is assessed according to the World Health Organization (WHO) criteria of severity, which are as follows:
mild: cases showing mild clinical symptoms with no sign of pneumonia on imaging.
moderate: cases showing fever and respiratory symptoms with radiological findings of pneumonia and requiring (O₂): 3 L/min<oxygen<5 L/min
severe: cases meeting any of the following criteria: respiratory distress (respiratory rate (RR)≥30 breaths/min); oxygen saturation (SpO₂)≤93% at rest in ambient air; or SpO₂≤97% with O₂>5 L/min; ratio of artery partial pressure of oxygen/inspired oxygen fraction (PaO₂/FiO₂)≤300 mmHg (1 mmHg=0.133 kPa), PaO₂/FiO₂ in high-altitude areas (at an altitude of over 1,000 meters above the sea level) shall be corrected by the following formula: PaO₂/FiO₂ [multiplied by][Atmospheric pressure (mmHg)/760]; and/or chest imaging that showed obvious lesion progression within 24-48 hours>50%.
critical: cases meeting any of the following criteria: respiratory failure and requiring mechanical ventilation; shock; and/or other organ failure that requires ICU care. In one embodiment, COVID-19 severity and/or progression is assessed with the WHO 10-point progression scale as indicated in Table 2 below.

TABLE 2
WHO 10-point progression scale of COVID-19

| WHO 10-point | | |
|---|---|---|
| Progression scale | Descriptor | Score |
| Uninfected | Uninfected; no viral RNA detected | 0 |
| Ambulatory | Asymptomatic; viral RNA detected | 1 |
| Ambulatory | Symptomatic; independent | 2 |
| Ambulatory | Symptomatic; assistance needed | 3 |
| Hospitalized: | Hospitalized; no oxygen therapy | 4 |
| mild disease | | |
| Hospitalized: | Hospitalized; oxygen by mask or nasal | 5 |
| mild disease | prongs | |
| Hospitalized: | Hospitalized; oxygen by non-invasive | 6 |
| severe disease | ventilation (NIV) or high flow | |
| Hospitalized: | Intubation and mechanical ventilation, | 7 |
| severe disease | PaO₂/FIO₂ ≥ 150 mmHg or oxygen saturation | |
| | to fraction of inspired oxygen ratio | |
| | (SpO₂/FIO₂) ≥ 200 mmHg | |
| Hospitalized: | Mechanical ventilation, PaO₂/FIO₂ < 150 | 8 |
| severe disease | mmHg or SpO₂/FIO₂ < 200 mmHg, or | |
| | vasopressors (norepinephrine > 0.3 μg/kg/min) | |
| Hospitalized: | Mechanical ventilation, PaO₂/FIO₂ < 150 | 9 |
| severe disease | mmHg and vasopressors (norepinephrine > 0.3 | |
| | μg/kg/min), or dialysis or ECMO | |
| Death | Dead | 10 |

In one embodiment, the subject to be treated according to the present invention is suffering from COVID-19 and has a score on the WHO 10-point progression scale of COVID-19 (as described in Table 2) ranging from 1 to 5, preferably ranging from 2 to 4; ranging from 3 to 6, preferably ranging from 4 to 5; or ranging from 5 to 9, preferably ranging from 6 to 8.

The compound or pharmaceutical composition of the invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration. In the treatment and/or prevention of an infectious disease an appropriate dosage level may be from about 0.01 to 500 mg per kg patient body weight per day (mg/kg/day), which can be administered in single or multiple doses. Typically, the dosage level will be from about 0.1 to about 250 mg/kg/day, preferably from about 0.5 to about 100 mg/kg/day, more preferably from about 2.5 to about 20 mg/kg/day. The compounds may be administered on a regimen of 1 to 4 times per day, preferably once or twice per day. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular diseases and the host undergoing therapy.

This invention also relates to the use of a compound of the invention as described herein, or a pharmaceutical composition of the invention as described herein, in the manufacture of a medicament for the treatment and/or prevention of an infectious disease. This invention also relates to a method for the treatment and/or prevention of an infectious disease in a subject in need thereof, comprising a step of administering to the subject a therapeutically effective amount of a compound of the invention as described herein, or of a pharmaceutical composition of the invention as described herein. This invention also relates to the use of a compound of the invention as described herein, or a pharmaceutical composition of the invention as described herein, in the treatment and/or prevention of an infectious disease.

### Glycocluster Synthesis

In the invention, the glycocluster may be prepared by any synthetic method known in the art. In particular, appropriate synthetic methods for creating a covalent linkage between the acetylated-sialic acid and macrocycle are part of the general knowledge of a person skilled in organic chemistry.

According to one embodiment, the acetylated-sialic acid may be linked to the macrocycle by means of at least one non-acetylated hydroxyl group on position 2, 4, 7, 8 or 9. In one embodiment, the acetylated-sialic acid is linked to the macrocycle by means of the hydroxyl on position 2 or 4. In one embodiment, the acetylated-sialic acid is linked to the macrocycle by means of the hydroxyl on position 2. The hydroxyl on position 2 is especially advantageous for linking the acetylated-sialic acid to the macrocycle without impairing the biological activity associated with the other side of the acetylated-sialic acid.

According to one embodiment, a non-acetylated hydroxyl group is used as a coupling function and reacted with a corresponding coupling function of the macrocycle (e.g., halocarbon, carboxylic acid or amine) to form a linkage between the acetylated-sialic acid and the macrocycle (e.g., an ether, ester or amide bond). According to another embodiment, the hydroxyl group is first substituted by a group comprising a coupling function other than hydroxyl and/or converted into a coupling function other than hydroxyl, then the other coupling function is reacted with a corresponding coupling function of the macrocycle to form a linkage between the acetylated-sialic acid and the macrocycle.

According to one embodiment, the glycocluster is prepared by Copper(I)-catalysed Azide-Alkyne Cycloaddition (CuAAC). In one embodiment, the glycocluster is prepared in presence of a copper (I) catalyst such as a copper (I) salt (e.g., copper bromide or copper iodide), or a mixture of a copper (II) salt (e.g., copper sulfate) and a reducing agent (e.g., sodium ascorbate).

According to one embodiment, the glycocluster is prepared by reaction of at least two equivalents of an acetylated-sialic acid comprising an azide (—N₃) with a macrocycle comprising at least two terminal alkynes (—C≡CH). In one embodiment, the acetylated-sialic acid comprises a —O—(CH₂)ₙ—N₃ group wherein n is an integer ranging from 1 to 8. In one embodiment, the macrocycle comprises at least two —O—(CH₂)ₘ—C≡CH groups wherein m is an integer ranging from 1 to 8.

According to another embodiment, the glycocluster is prepared by reaction of at least two equivalents of an acetylated-sialic acid comprising a terminal alkyne (—C≡CH) with a macrocycle comprising at least two azide (—N₃). In one embodiment, the acetylated-sialic acid comprises a —O—(CH₂)ₙ—C≡CH group wherein n is an integer ranging from 1 to 8. In one embodiment, the macrocycle comprises at least two —O—(CH₂)ₘ—N₃ groups wherein m is an integer ranging from 1 to 8.

### BRIEF DESCRIPTION OF THE DRAWINGS

FIG. 1 is a scheme showing the method of preparation of the SA- or 9-AcSA-derived glycoclusters 004-014 from α-propargyl sialic acid (α-p-SA) 001 or α-propargyl acetylated sialic acid (α-p-9-AcSA) 002 and azide compounds 003, 006, 009 and 012 (which are shown in Scheme 3 in Example 1-d).

FIG. 2 is a box plot showing the specific binding probabilities (BP) measured between the S1 functionalized tip and the surface coated with 9-AcSA, SA or streptavidin, before and after blocking with free 1 mM 9-AcSA, as described in Example 2.1. One data point represents the binding frequency (BF) obtained for 1024 FD curves. The square in the box indicates the mean, the coloured box the 25^{th} and 75^{th} percentiles, and the whiskers the highest and lowest values. The line in the box indicates the median. N=9 maps examined over 3 independent experiments. P-values were determined by two-sample t test in Origin.

FIG. 3 is a graph showing the binding frequency (BF) plotted between S1 and 9-AcSA as a function of the hold time, as described in Example 2.2. Least-squares fits of the data to a mono-exponential decay curve (line) provides average kinetic on-rates (kₒₙ) of the probed interaction. Further calculation (k_{off}/kₒₙ) leads to K_{D}. One data point represent the BF obtained for 1024 FD curves.

FIGS. 4 to 9 show the results of a screening of the anti-binding properties of SA- or 9-AcSA-derived glycoclusters, as described in Example 2.5.

FIGS. 4-8 are histograms showing the inhibiting efficiency of the tested molecules, which is evaluated by measuring the binding probability (BP) of the interaction between 9-AcSA and SARS-CoV-2 before and after incubation with the tested molecules at increasing concentration (1-100 μM). Normalized histograms show the relative BP of the interaction between 9-AcSA and SARS-CoV-2 before and after incubation with 1, 10, or 100 μM of free acetylated sialic acid (9-AcSA) (FIG. 4), SA- and 9-AcSA-pillar[5]arenes 004 and 005 (FIG. 5), SA- and 9-AcSA-fullerenes 013 and 014 (FIG. 6), SA- and 9-AcSA-porphyrins 010 and 011 (FIG. 7) or SA- and 9-AcSA-calix[4]arenes 007 and 008 (FIG. 8).

FIG. 9 is a graph showing the reduction of the binding probability (BP) after incubation with the acetylated tested molecules 9-AcSA-α-p, 005, 008, 011 and 014 at increasing concentration (1-100 μM) as described in Example 2.5. Data are representative of at least N=3 independent experiments (tips and sample) per SA dendrimer concentration. P-values were determined by two-sample t test in origin. The error bars indicate standard deviation (s.d.) of the mean value.

FIGS. 10 and 11 show the results of a probing of 9-AcSA-porphyrin glycocluster 011 efficiency to inhibit SARS-CoV-2 binding to acetylated SA on model surfaces (9-O-acetylated SA) and living cells (CHO-cells) at low concentrations, as described in Example 2.6.

FIG. 10 is a box plot showing the relative binding values of the interaction between SARS-Cov-2 and 9-O-acetylated SA model surfaces before and after incubation with 9-AcSA-porphyrin glycocluster 011 at increasing concentration (0.001-100 μM).

FIG. 11 is a box plot showing the relative binding values of the interaction between SARS-Cov-2 and CHO-cells before and after incubation with 9-AcSA-porphyrin glycocluster 011 at increasing concentration (0.1-10 μM).

FIG. 12 is a histogram showing the results of an infectivity assay, namely, the infectivity measured in the presence of free SA, free 9-AcSA, SA-porphyrin 010 and 9-AcSA porphyrin 011, as described in Example 2.7. Each dot shows the infectivity from a well. The colored box indicates the mean and the whiskers the s.d. of the mean value. The line in the box indicates the median. P values were determined by two-sample t test in Origin.

FIGS. 13A-B are two box plots of the binding probability (BP, in %) between S1 and Lec2 or CHO cells (FIG. 13A) or between SARS-CoV-2 and Lec2 or CHO cells (FIG. 13B). n=10 (FIG. 13A) or 12 (FIG. 13B) maps examined over three independent experiments. P values were determined by two-sample t-test in Origin.

### EXAMPLES

The present invention is further illustrated by the following examples.

### Example 1: Materials, Methods and Results for Synthesis and Characterization

### Example 1-a: General Materials and Methods

The solvents used for chromatography were purchased in industrial grade and further distilled before their use. Reagents and chemicals were purchased from Sigma-Aldrich or Acros at ACS grade and were used without purification.

All reactions were monitored by thin-layer chromatography (TLC) carried out on Merck aluminium roll silica gel 60-F₂₅₄ using KMnO₄ and a phosphomolybdic acid solution as developers. Merck silica gel (60, particle size 40-63 μm) was employed for flash column chromatography. NMR spectra were recorded on a JEOL ECX 400 or 500 with solvent peaks as reference. The compounds were characterized by ¹H and ¹³C NMR as well as by ¹H-¹H and ¹H-¹³C correlation experiments. The abbreviations used to define the multiplicities are: s=singlet, d=doublet, t=triplet, q=quadruplet, m=multiplet and br=broad. Chemical shifts (δ) are reported in ppm and referenced indirectly to the residual solvent signals. High resolution mass spectra (HRMS) were carried out on the Bruker maXis Impact QTOF mass spectrometer and Bruker MicroTOF-Q II XL spectrometer.

### Example 1-b: Synthesis and Characterization of Biotinylated Sialic Acids

Compounds B4 and B5 were prepared as shown in Scheme 1.

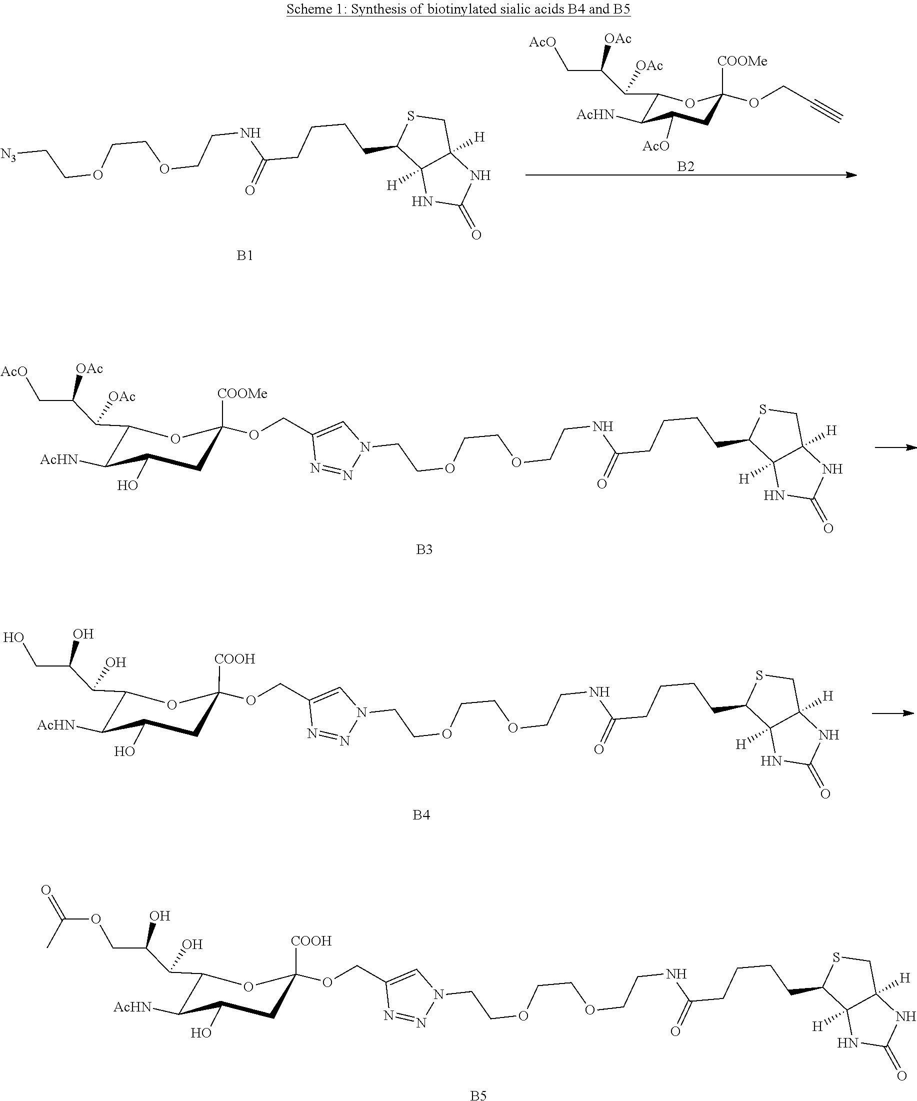

Intermediate compound B1 was prepared according to known procedures (Jung, D. et al. Chemical Communications, 2014, Vol. 50, pp. 3044-3047). Intermediate compound B2 was prepared according to known procedures (Gan, Z. & Roy, R., Canadian Journal of Chemistry, 2002, Vol. 80, pp. 908-916). Compound B5 was acetylated following a method previously described (Ogura, H. et al. Carbohydrate Research, 1987, Vol. 167, pp. 77-86).

Intermediate compound (B3): To a solution of compound B1 (401 mg, 1.0 mmol, 1 equiv.), compound B2 (636 mg, 1.2 mmol, 1.2 equiv.) and Et₃N (0.278 mL, 2.0 mmol, 2 equiv.) in dry dimethylformamide (DMF) (20 mL) was added CuI (38 mg, 0.2 mmol, 0.2 equiv.) under argon atmosphere. The solution was stirred for 12 h at room temperature. After having evaporated the solvent under vacuum, EtOAc (30 mL) and saturated ammonium chloride (30 mL) were added, and the phases separated. The organic layer was washed with brine (30 mL), dried over MgSO₄, filtered and the filtrate was concentrated under reduced pressure. The residue was purified by column chromatography on silica gel using dichloromethane (DCM)/MeOH (20:1) as eluent to give a white solid (975 mg, 1.15 mmol, 96% yield). ¹H NMR (500 MHz, CDCl₃) 7.79 (s, 1H, H-12), 6.68 (br, 1H, NH), 6.43 (br, 1H, NH), 5.47 (d, J=46.4 Hz, 1H, I0a), 5.45-5.42 (m, H-8), 5.35-5.32 (m, 1H, H-7), 4.92-4.87 (m, 2H, H-4, H-10b), 4.60-4.53 (m, 4H, H-13, H-17), 4.38-4.33 (m, 2H, H-9a, H-25), 4.16-4.05 (m, 3H, H-5, H-6, H-9b), 3.91 (t, J=5.1 Hz, 2H, H-14), 3.80 (s, 3H, COCH₃), 3.61 (dd, J=6.0, 2.7 Hz, 2H, H-15), 3.56 (dd, J=5.9, 2.7 Hz, 2H, H-15), 3.50 (t, J=5.2 Hz, 2H, H-17), 3.41-3.40 (m, 2H, H-18), 3.17 (dd, J=11.8, 7.3 Hz, 1H, H-24), 2.95-2.88 (m, 1H, H-28a), 2.77 (d, J=12.9 Hz, 1H, H-3a), 2.62 (dd, J=12.8, 4.6 Hz, 1H, H-28b), 2.24 (t, J=7.5 Hz, 2H, H-20), 2.16 (s, 3H, OCOCH₃), 2.15 (s, 3H, OCOCH₃), 2.03 (s, 3H, OCOCH₃), 2.02 (s, 3H, OCOCH₃), 1.97 (t, J=12.5 Hz, 1H, H-3b), 1.88 (s, 3H, NCOCH₃), 1.77-1.63 (m, 4H, H-21, H-23), 1.48-1.42 (m, 2H, H-22). ¹³C NMR (126 MHz, CDCl₃) δ 173.5 (C-19), 171.0, 170.80, 170.5, 170.3, 170.2 (C═O, NCOCH₃, OAc), 168.2 (C-1), 164.0 (Cq, C-26), 143.8 (C-11), 124.4 (C-12), 98.6 (C-2), 72.8 (C-6), 70.4 (C-16), 70.1 (C-17), 69.9 (C-15), 69.4 (C-14), 69.2 (C-4), 68.5 (C-8), 67.5 (C-7), 62.6 (C-9), 61.9 (C-25), 60.3 (C-27), 58.4 (C-10), 55.7 (C-24), 53.0 (OCH₃), 50.2 (C-13), 49.2 (C-5), 40.5 (C-28), 39.1 (C-18), 37.9 (C-3), 35.9 (C-20), 28.3 (C-22), 28.1 (C-23), 25.6 (C-21), 23.2 (NCOCH₃), 21.2, 21.0, 2×20.9 (OCOCH₃). HRMS (TOF-MS-ESI⁺, m/z): calculated for C₃₉H₆₀N₇O₁₇S [M+H]⁺ 930.3761; found 930.3750.

Compound (B4) (“biot-SA”): A solution of compound B3 (345 mg, 0.371 mmol, 1 equiv.) and NaOMe (40 mg, 0.742 mmol, 2 equiv.) in dry MeOH (20 mL) was stirred at 0° C. for 30 min, then warmed up to room temperature and stirred for another 1.5 h. Afterwards, Amberlyst®15 ion-exchange resin was added to neutralize the base. The resin was filtered and washed with water (2×5 mL). The filtrate was evaporated under reduced pressure to afford a white solid. Without further purification, the white solid was dissolved in water (15 mL) and LiOH·H₂O (35 mg, 0.831 mmol, 3 equiv.) was added. The solution was stirred at room temperature for 1 h before adding Amberlyst®15 ion-exchange. Afterwards, the reaction mixture was filtered, the resin was washed with water (2×5 mL) and the filtrate was lyophilized to obtain white solid (186 mg, 0.249 mmol, 90% yield). ¹H NMR (500 MHz, D₂O) δ 8.06 (s, 1H, H-12), 4.92-4.89 (m, 1H, H-10a), 4.68-4.66 (m, 1H, H-10b), 4.62-4.60 (m, 2H, H-13), 4.57-4.53 (m, 1H, H-27), 4.38-4.34 (m, 1H, H-25), 3.95-3.93 (m, 2H, H-14), 3.88-3.79 (m, 4H, H-5, H-8, H-6, H-9a), 3.76-3.71 (m, 1H, H-4), 3.65-3.55 (m, 6H, H-15, H-16, H-9b, H-7), 3.52-3.49 (m, 2H, H-17), 3.32-3.30 (m, 2H, H-18), 3.27-3.24 (m, 1H, H-24), 2.95-2.90 (m, 1H, H-28a), 2.74-2.68 (m, 2H, H-3a, H-28b), 2.21 (dd, J=13.2, 6.5 Hz, 2H, H-20), 2.01-2.00 (m, 3H, NAc), 1.74 (t, J=12.2 Hz, 1H, H-3b), 1.61-1.48 (m, 4H, H-21, H-23), 1.36-1.33 (m, 2H, H-22). ¹³C NMR (126 MHz, D₂O) δ 176.8 (C═O, NCOCH₃), 175.0 (C-1), 171.8 (C═O, C-19), 165.3 (C═O, C-26), 143.6 (C-11), 125.7 (C-12), 99.5 (C-2), 72.9 (C-6), 71.1 (C-8), 69.7 (C-16), 69.3 (C-17), 68.9 (C-15), 68.7 (C-14), 68.3 (C-7), 67.7 (C-4), 62.9 (C-9), 62.1 (C-25), 60.3 (C-27), 57.2 (C-10), 55.4 (C-24), 51.8 (C-5), 50.1 (C-13), 2×39.7 (C-28, C-3), 38.9 (C-18), 35.5 (C-20), 27.9 (C-22), 27.7 (C-23), 25.2 (C-21), 22.1 (NCOCH₃). HRMS (TOF-MS-ESI⁺, m/z): calculated for C₃₀H₅₀N₇O₁₃S [M+H]⁺ 748.3182; found 748.3173.

Compound (B5) (“biot-9-AcSA”): To a solution of compound B4 (200 mg, 0.267 mmol, 1 equiv.) and trimethyl orthoacetate (0.34 mL, 2.67 mmol, 10 equiv.) in dry dimethylsulfoxide (DMSO) (1.2 mL) was added p-toluenesulfonic acid monohydrate (5.0 mg, 0.027 mmol, 0.1 equiv.). The solution was stirred at room temperature for 12 h. Then, DCM (50 mL) was added to precipitate the crude product. The crude was purified by Cis silica gel flash chromatography using H₂O/MeOH (0-⅓, gradient) as eluent. The fractions containing compound B5 were combined and concentrated under reduced pressure. The concentrated solution was lyophilized to afford the desired compound as a white solid (25 mg, 0.0316 mmol, 12%). ¹H NMR (500 MHz, CD₃OD) δ 8.02 (s, 1H, H-12), 4.94 (d, J=12.1 Hz, 1H, H-10a), 4.67 (d, J=12.1 Hz, 1H, H-10b), 4.55-4.55 (m, 2H, H-13), 4.49 (dd, J=7.8, 4.7 Hz, 1H, H-27), 4.38 (dd, J=11.2, 1.8 Hz, 1H, H-25), 4.31 (dd, J=7.9, 4.5 Hz, 1H, H-9a), 4.14-4.06 (m, 2H, H-8, H-9b), 3.90 (t, J=5.1 Hz, 2H, H-14), 3.74-3.71 (m, 2H, H-5, H-4), 3.64-3.60 (m, 3H, H-6, H-16), 3.58-3.56 (m, 2H, H-17), 3.51-3.48 (m, 3H, H-7, H-15), 3.35-3.33 (m, 2H, H-18), 3.20-3.17 (m, 1H, H-24), 2.94-2.84 (m, 2H, H-3a, H-28a), 2.69 (d, J=12.7 Hz, 1H, H-28b), 2.21 (t, J=7.4 Hz, 2H, H-20), 2.05 (s, OCOCH₃), 2.02 (s, NCOCH₃), 1.74-1.71 (m, 1H, H-3b), 1.66-1.5 (m, 4H, H-21, H-23), 1.45-1.41 (m, 2H, H-22). ¹³C NMR (126 MHz, CD₃OD) δ 176.2 (C═O, NCOCH₃), 175.5 (C-1), 174.0 (C═O, 9-O—Ac), 173.1 (C═O, C-19), 166.1 (C═O, C-26), 146.5 (C-11), 125.9 (C-12), 101.9 (C-2), 74.2 (C-6), 71.4 (C-16), 71.2 (C-17), 70.7 (C-8), 2×70.6 (C-7, C-15), 70.3 (C-14), 69.6 (C-4), 67.3 (C-9), 63.3 (C-25), 61.6 (C-27), 58.9 (C-10), 57.0 (C-24), 54.1 (C-5), 51.3 (C-13), 42.6 (C-3), 41.1 (C-28), 40.3 (C-18), 36.7 (C-20), 29.7 (C-22), 29.5 (C-23), 26.8 (C-21), 22.6 (NCOCH₃), 20.8 (OCOCH₃). HRMS (TOF-MS-ESI⁺, m/z): calculated for C₃₂H₅₂N₇O₁₄S [M+H]⁺ 790.3287; found 790.3287.

### Example 1-c: Synthesis and Characterization of Sialic Acid Derivatives

Intermediate compound 001 was prepared according to known procedures (Daskhan, G. et al., ChemistryOpen, 2016, Vol. 5, pp. 477-484). Intermediate compound 002 was prepared according to known procedures (Ogura, H. et al., Carbohydrate Research, 1987, Vol. 167, pp. 77-86), as shown in Scheme 2.

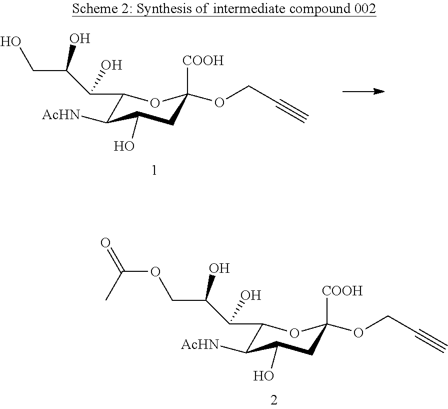

Prop-2-ynyl 5-acetamido-9-O-acetyl-3,5-dideoxy-D-glycero-α-D-galacto-2-onulopyranose (002): To a solution of compound 001 (380 mg, 1.1 mmol, 1 equiv.) and trimethyl orthoacetate (1.4 mL, 11 mmol, 10 eq.) in dry DMSO (4 mL) was added p-toluenesulfonic acid monohydrate (10 mg, 0.05 mmol, 0.05 equiv.). The solution was stirred at room temperature for 12 h. Then, DCM (20 mL) was added to precipitate the crude product. After a slow cotton filtration, the solid on the filtrate was re-dissolved in methanol and transferred to evaporate the solvent. The residue was purified by silica gel chromatography using DCM/MeOH (20:3) to afford the desired compound (2) as a white solid (167 mg, 0.429 mmol, 39% yield).

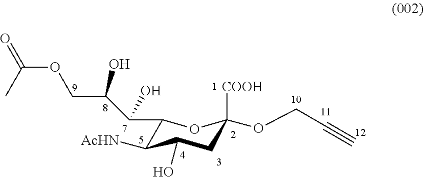

¹H NMR (500 MHz, CD₃OD) δ: 4.42-4.35 (m, 2H, H-9a, H-10a), 4.17-4.03 (m, 3H, H-10b, H-9b, H-8), 3.74-3.65 (m, 2H, H-4, H-5), 3.57-3.54 (m, 1H, H-6), 3.49 (dd, J=9.2, 1.9 Hz, 1H, H-7), 2.83 (dd, J=12.3, 4.4 Hz, 1H, H-3a), 2.75 (t, J=2.4 Hz, 1H, H-13), 2.06 (s, 3H, OCOCH₃), 2.03 (s, 3H, NHCOCH₃), 1.60 (t, J=11.7 Hz, 1H, H-3b). ¹³C NMR (126 MHz, CD₃OD) δ 175.6 (C═0, NAc), 173.5 (C-1), 173.1 (C═O, 9-O—Ac), 101.6 (C-2), 80.6 (C-11), 75.0 (C-12), 74.1 (C-6), 70.6 (C-8), 70.5 (C-7), 69.2 (C-4), 67.2 (C-9), 54.0 (C-5), 53.1 (C-10), 42.2 (C-3), 22.7 (NCOCH₃), 20.8 (OCOCH₃). HRMS (TOF-MS-ESI⁺, m/z): calculated for C₁₆H₂₄NO₁₀ [M+H]⁺ 390.1395; found 390.1395.

### Example 1-d: Synthesis and Characterization of Glycoclusters

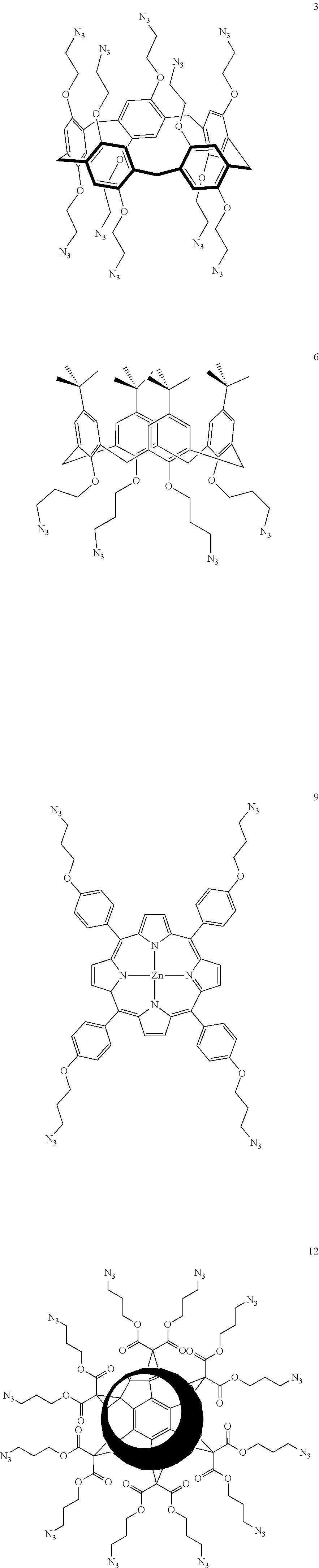

### Scheme 3: Structures of Intermediate Compounds 003, 006, 009 and 012

Intermediate compound 003 was prepared according to known procedures (Nierengarten, I. et al., Chemical Communications, 2012, Vol. 48, pp. 8072-8074). Intermediate compound 006 was prepared according to known procedures (Tikad, A. et al., Chemistry: A European Journal, 2016, Vol. 22, pp. 13147-13155). Intermediate compound 009 was prepared according to known procedures (Tikad, A. et al., Chemistry: A European Journal, 2016, Vol. 22, pp. 13147-13155; and Liu, Y. et al., Angewandte Chemie (International Edition English), 2016, Vol. 55, pp. 7952-7957). Intermediate compound 012 was prepared according to known procedures (Nierengarten, J. F. et al., Chemical Communications, 2010, Vol. 46, pp. 3860-3862).

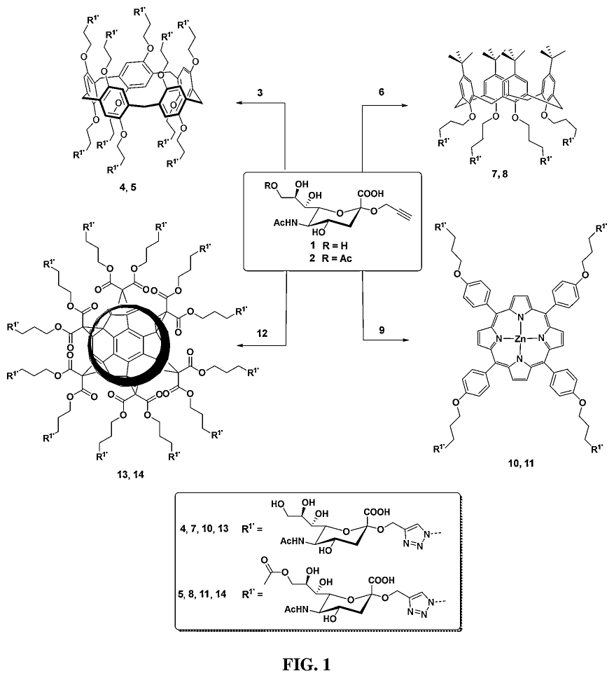

Compounds 004, 007, 010 and 013 (“SA-glycoclusters”) and 005, 008, 011 and 014 (“9-As-SA-glycoclusters”) were prepared by grating the clickable α-propargyl sialic acids 001 and 002 to multimeric azides 003, 006, 009 and 0012 using either a combination of copper(II)sulfate and sodium L-ascorbate, or copper(I)bromide dimethyl sulphide, as shown in FIG. 1.

More specifically, an excess amount of 001 or 002 was coupled to tetra-azide pillar[5]arene 003 with a catalytic amount of copper(II)sulfate, and sodium L-ascorbate in a mixed solvent system (1,4-dioxane/H₂O, 2:1) at room temperature overnight. Then, 004 and 005 were precipitated with acetone and purified by copper scavenging (Quadrasil MP) and size-exclusion Sephadex®G-25 chromatography. The purified decavalent pillar[5]arenes 004 and 005 were obtained in 74% and 62% yield, respectively. A specific method was optimized for the porphyrin tetramers 010 and 011, to avoid ion exchange between copper and zinc and to cope with the solubility properties of 009. Thus, a lower catalyst amount and a ternary solvent system (THF/DMSO/H₂O, 3:3:1) were employed. The tetravalent porphyrin conjugates 010 and 011 were obtained in 61% and 87% yields, respectively. The calix[4]arenes 007 and 008 and fullerenes 013 and 014 were also obtained in high yields using similar coupling and purification protocols. All the multimeric species were characterized by ¹H NMR, ¹³C NMR and mass spectrometry to confirm the completion of all reactions.

Compound (004): To a solution of compound 003 (21 mg, 0.01 mmol, 1 equiv.) and compound 001 (70 mg, 0.20 mmol, 12 equiv.) in 1,4-dioxane (2 mL) was added a freshly prepared solution of CuSO₄ (3.2 mg, 0.02 mmol, 2 equiv.) and NaAsc (13 mg, 0.066 mmol, 6.6 equiv.) in H₂O (1 mL) under argon atmosphere. The reaction mixture was vigorously stirred overnight at room temperature. Then acetone (40 mL) was added to precipitate the crude product. After centrifugation, the crude was dissolved in water (2 mL) and treated with Quadrasil MP (40 mg) to remove the residual copper ions. After filtration through a 45 μm sterile filter, the filtrate was passed through a Sephadex® G-25 column and eluted with water. The fractions that could not migrate by TLC elution with DCM/MeOH (1:0.3, KMnO₄ staining) were collected. The combined fractions were lyophilized to afford a white solid (59 mg, 0.0124 mmol, 74% yield).

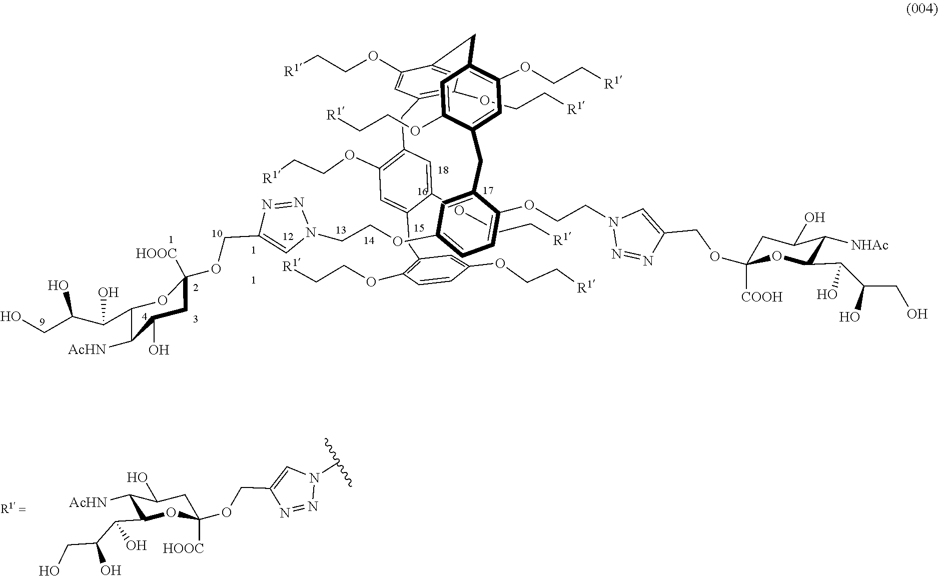

¹H NMR (500 MHz, DMSO-d₆) δ 8.17-8.06 (m, 10H, 10×H-12), 6.57 (s, 10H, 10×H-16), 4.77-4.13 (m, 60H, 10×H-10, 10×H-13, 10×H-14), 3.61-3.31 (m, 80H, 10×H-4, 10×H-5, 10×H-6, 10×H-7, 10×H-8, 10×H-9, 10×H-18), 2.63 (s, 10H, 10×H-3a), 1.87 (s, 30H, 10×NCOCH₃), 1.43 (s, 10H, 10×H-3b). ¹³C NMR (126 MHz DMSO-d₆) δ 173.9 (C═O, NCOCH₃), 172.0 (C-1), 149.7 (Cq, C-15), 145.2 (Cq, C-11), 129.6 (Cq, C-17), 125.2 (C-12), 116.1 (C-16), 100.5 (C-2), 73.5 (C-6), 72.1 (C-8), 69.3 (C-7), 68.1 (C-4, C-14), 63.6 (C-9), 57.8 (C-10), 53.2 (C-5), 50.8 (C-13), 41.6 (C-3), 29.0 (C-18), 23.2 (NCOCH₃). HRMS (TOF-MS-ESI⁻, m/z): calculated for C₁₉₅H₂₆₇N₄₀O₁₀₀ [M-3H]³⁻ 1590.2365; found 1590.2262.

Compound (005): To a solution of compound 003 (13 mg, 0.01 mmol, 1 equiv.) and compound 002 (47 mg, 0.12 mmol, 12 equiv.) in 1,4-dioxane (1.2 mL) was added a freshly prepared solution of CuSO₄ (3.2 mg, 0.02 mmol, 2 equiv.) and NaAsc (13 mg, 0.066 mmol, 6.6 equiv.) in H₂O (0.6 mL) under argon atmosphere. The reaction mixture was vigorously stirred overnight at room temperature. Then, acetone (20 mL) was added to precipitate the crude product. After centrifugation, the crude was dissolved in water (2 mL) and treated with Quadrasil MP (40 mg) to remove the residual copper ions. After filtration through a 45 μm sterile filter, the filtrate was passed through a Sephadex®G-25 column and eluted with water. The fractions that could not migrate by TLC elution with DCM/MeOH (1:0.3) and KMnO₄ staining were collected. The combined fractions were lyophilized to afford a white solid (32 mg, 0.0062 mmol, 62% yield).

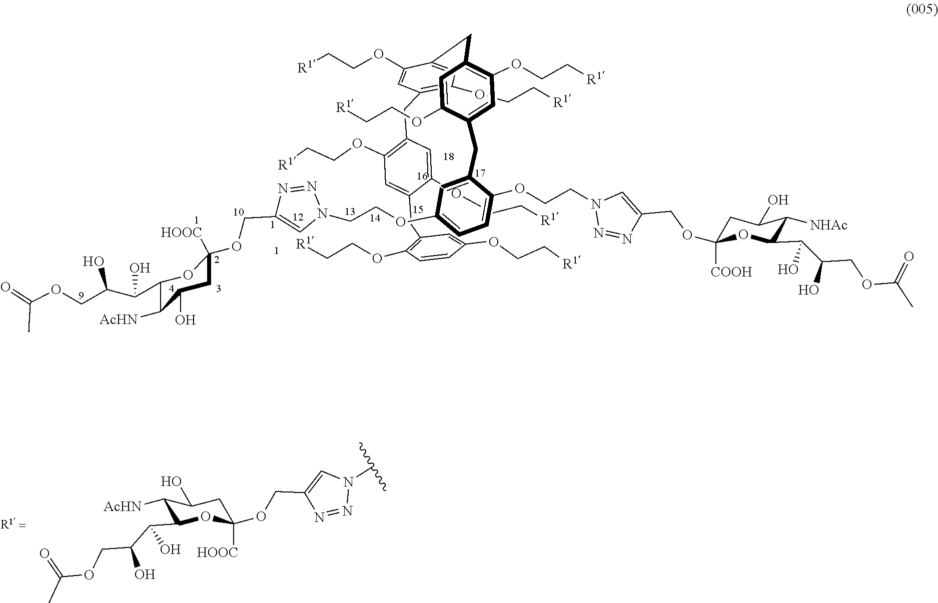

¹H NMR (500 MHz, CD₃OD) δ 8.16 (br, 10H, 10×H-12), 6.62 (br, 10H, 10×H-16), 4.69 (br, 30H, 10×H-10b, 10×H-13), 4.37 (br, 20H, 10×H-9), 4.09 (br, 30H, 10×H-8, 10×H-14), 3.76 (br, 40H, 10×H-4, 10×H-5, 10×H-6, 10×H-18), 3.53 (br, 10H, 10×H-7), 2.83 (br, 10H, 10×H-3a), 2.03 (br, 60H, 10×OCOCH₃, 10×NCOCH₃), 1.65 (br, 10H, 10×H-13b). ¹³C NMR (126 MHz, CD₃OD) δ 175.3 (C═O, NCOCH₃), 173.9 (C-1), 173.3 (C═O, 9-O—Ac), 151.0 (Cq, C-15), 146.3 (Cq, C-11), 130.1 (Cq, C-17), 126.0 (C-12), 117.1 (C-16), 101.7 (C-2), 74.3 (C-6), 70.6 (C-8), 69.3 (C-7), 68.6 (C-4, C-14), 67.3 (C-9), 58.9 (C-10), 54.0 (C-5), 51.4 (C-13), 42.1 (C-3), 31.0 (C-18), 22.9 (NCOCH₃), 21.0 (OCOCH₃). HRMS (TOF-MS-ESI⁺, m/z): calculated for C₂₁₅H₂₉₃N₄₀O₁₁₀ [M+3H]³⁺ 1732.2869; found 1732.2863.

Compound (007): To a solution of compound 006 (36 mg, 0.03 mmol, 1 equiv.) and compound 001 (46 mg, 0.132 mmol, 4.4 equiv.) in dry DMSO (1 mL) was added CuBr·CH₃SCH₃ (3.7 mg, 0.018 mmol, 0.6 equiv.) under argon atmosphere. After 4 h under microwave irradiations at 80° C., the crude was cooled down and precipitated by adding DCM (30 mL). The precipitate was then dissolved in water (2 mL) and treated with Quadrasil®MP (70 mg) to remove the residual copper ions. After filtration through a 45 μm sterile filter, the filtrate was passed through a Sephadex© G-15 column and eluted with water. The fractions that could not migrate by TLC elution with DCM/MeOH (1:0.3) and KMnO₄ staining were collected. The combined fractions were lyophilized to afford a white solid (70 mg, 0.0276 mmol, 92% yield).

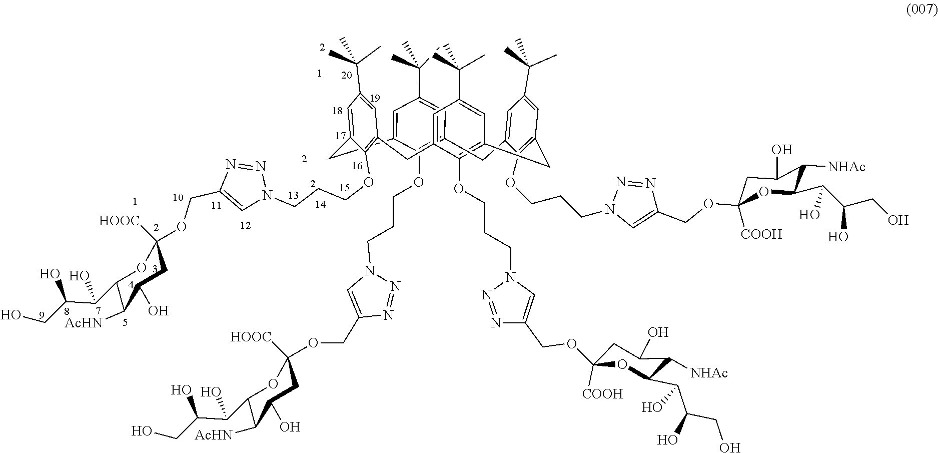

¹H NMR (500 MHz, CD₃OD) δ 8.03 (s, 4H, 4×H-12), 6.82 (s, 8H, 8×H-18), 4.62 (br, 8H, 8×H-13), 4.35 (br, 4H, 4×H-22a), 3.89-3.58 (m, 52H, 4×H-4, 4×H-5, 4×H-6, 4×H-7, 4×H-8, 4×H-9, 4×H-15, 16×OH), 3.13 (br, 4H, 4×H-22b), 2.82 (br, 4H, 4×H-3a), 2.52 (br, 8H, 4×H-14), 2.02 (s, 12H, 4×NCOCH₃), 1.66 (br, 4H, 4×H-3b), 1.08 (s, 36H, 12×H-21). ¹³C NMR (126 MHz, CD₃OD) δ 175.3 (C═O, NCOCH₃), 173.9 (C-1), 154.2 (Cq, C-16), 146.1 (Cq, C-11, C-19), 134.9 (Cq, C-17), 126.4 (C-18), 125.6 (C-12), 101.3 (C-2), 74.50 (C-6), 73.0 (C-15), 70.1 (C-7, C-8), 69.2 (C-4), 64.3 (C-9), 58.4 (C-10), 53.9 (C-5), 48.5 (C-13), 41.9 (C-3), 34.7 (Cq, C-20), 32.0 (C-14, C-21, C-22), 22.9 (NCOCH₃). HRMS (TOF-MS-ESI⁺, m/z): after deconvolution calculated for C₁₂NH₁₇₀N₁₆O₄₄ [M] 2369.0978; found 2369.0877.

Compound (008): To a solution of compound 006 (37 mg, 0.03 mmol, 1 equiv.) and compound 002 (51 mg, 0.132 mmol, 4.4 equiv.) in dry DMSO (1 mL) was added CuBr·CH₃SCH₃ (3.7 mg, 0.018 mmol, 0.6 equiv.) under argon atmosphere. After 4 h under microwave irradiations at 80° C., the crude was precipitated by adding DCM (30 mL). The precipitate was then dissolved in water (2 mL) and treated with Quadrasil®MP (70 mg) to remove the residual copper ions. After filtration through a 45 m sterile filter, the filtrate was passed through a Sephadex® G-15 column and eluted with water. The fractions that could not migrate by TLC elution with DCM/MeOH (1:0.3, KMnO₄ staining) were collected. The combined fractions were lyophilized to afford a white solid (62 mg, 0.0244 mmol, 81% yield).

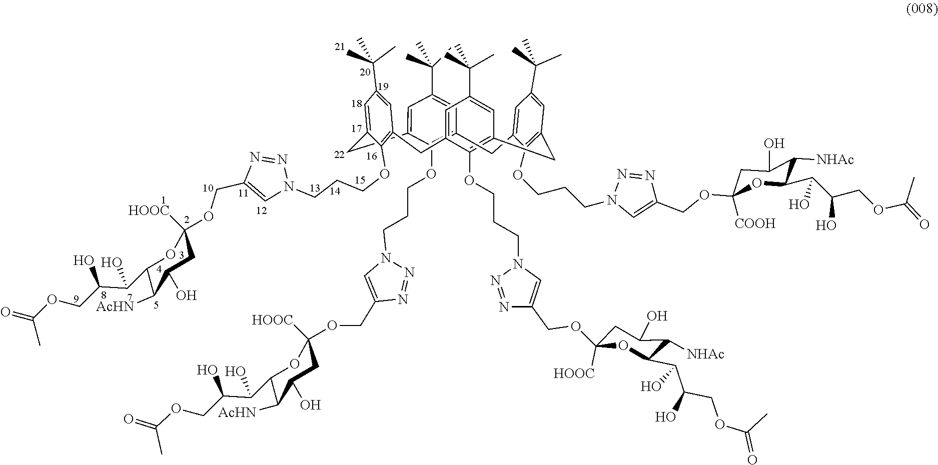

¹H NMR (500 MHz, CD₃OD) δ 8.21-8.06 (m, 4H, 4×H-12), 6.82 (s, 8H, 8×H-18), 4.63 (br, 8H, 4×H-13), 4.37 (s, 8H, 4×H-9a, 4×H-22a), 4.13 (br, 8H, 4×H-8, 4×H-9b), 3.88-3.52 (m, 24H, 4×H-4, 4×H-5, 4×H-6, 4×H-7, 4×H-15), 3.13 (s, 4H, 4×H-22b), 2.79 (br, 4H, 4×H-3a), 2.53 (br, 8H, 4×H-14), 2.02 (br, 24H, 4×NCOCH₃, 4×OCOCH₃), 1.70 (br, 4H, 4×H-3b), 1.33 (s, 6H, 2×H-21), 1.08 (s, 24H, 8×H-21), 0.85 (s, 6H, 2×H-21). ¹³C NMR (126 MHz, CD₃OD) δ 175.3 (C═O, NCOCH₃), 173.1 (C═O, C-1, 9-O—Ac), 154.2 (Cq, C-16), 146.4 (Cq, C-11), 146.0 (Cq, C-19), 134.9 (Cq, C-17), 126.4 (Cq, C-12, C-18), 100.8 (C-2), 74.4 (C-6), 72.9 (C-15), 70.5 (C-8), 70.4 (C-7), 69.1 (C-4), 67.4 (C-9), 58.7 (C-10), 53.8 (C-5), 42.0 (C-3), 34.7 (Cq, C-20), 32.0 (C-14, C-21, C-22), 22.75 (OCOCH₃), 20.9 (OCOCH₃). HRMS (TOF-MS-ESI⁺, m/z): after deconvolution calculated for C₁₂₀NH₁₇₀N₁₆O₄₄ [M] 2537.1400; found 2537.1298.

Compound (010): To a solution of compound 009 (40 mg, 0.037 mmol, 1 equiv.) and compound 001 (57 mg, 0.164 mmol, 4.4 equiv,) in THF/DMSO (1.5 mL, 2:3) was added a freshly prepared solution of CuSO₄ (2.36 mg, 0.0148 mmol, 0.4 equiv.) and NaAsc (8.7 mg, 0.0439 mmol, 1.2 equiv.) in H₂O (0.3 mL) under argon atmosphere. The reaction mixture was vigorously stirred overnight at room temperature. Then, acetone (30 mL) was added to precipitate the crude product. The crude was then dissolved in water (2 mL) and treated with Quadrasil®MP (80 mg) to remove the residual copper ions. After filtration through a 45 μm sterile filter, the filtrate was passed through a Sephadex® G-15 column and eluted with water. The fractions that could not migrate by TLC elution with DCM/MeOH (1:0.3, KMnO₄ staining) were collected. The combined fractions were lyophilized to afford a dark green solid (56 mg, 0.0227 mmol, 61% yield).

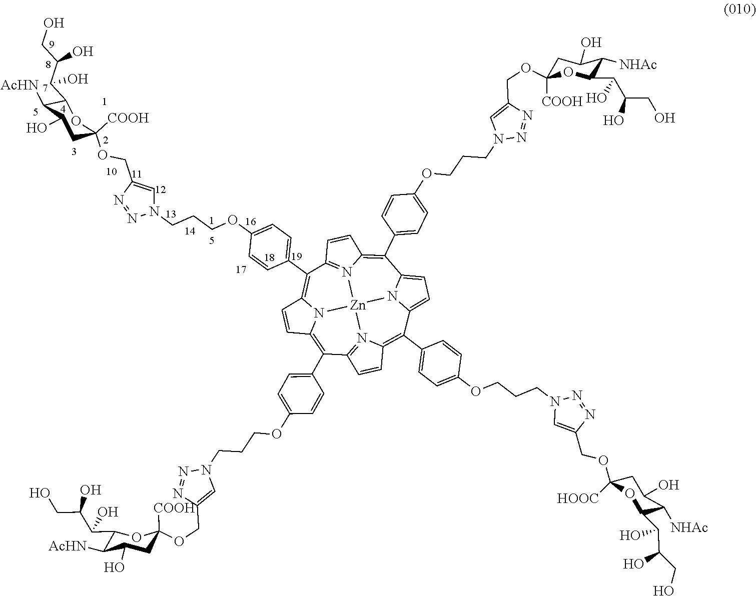

¹H NMR (400 MHz, D₂O/DMSO-d₆) δ 9.07 (br, 8H, 8×H-_{Porph}), 8.32 (br, 12H, 4×H-12, 8×H-18), 7.51 (br, 8H, 8×H-17), 3.74 (br, 28H, 4×H-4, 4×H-5, 4×H-6, 4×H-7, 4×H-8, 4×H-9), 2.78 (br, 12H, 4×H-14, 4×H-3a), 2.05 (br, 16H, 4×H-3b, 4×NCOCH₃). ¹³C NMR (101 MHz, D₂O/DMSO-d₆) δ 176.3 (C═O, NCOCH₃), 173.9 (C-1), 159.5 (C-16), 151.3 (Cq, C-19), 146.6 (Cq, C-11), 137.2 (C-18), 135.5 (Cq, C-_{Porph}), 133.3 (C-_{Porph}), 127.0 (C-12), 121.5 (Cq, C-_{Porph}), 114.5 (C-17), 101.9 (C-2), 74.3 (C-6), 73.1 (C-8), 69.7 (C-4, C-7), 67.0 (C-15), 64.1 (C-9), 59.1 (C-10), 53.4 (C-5), 49.4 (C-13), 41.8 (C-3), 30.9 (C-14), 23.6 (NCOCH₃). HRMS (TOF-MS-ESI⁺, m/z): calculated for C₁₁₂H₁₃₄N₂₀O₄₀Zn [M+2H]²⁺ 1232.4186; found 1232.4390.

Compound (011): To a solution of compound 009 (40 mg, 0.037 mmol, 1 equiv.) and compound 002 (63 mg, 0.162 mmol, 4.4 equiv,) in THF/DMSO (1.8 mL, 1:1) was added a freshly prepared solution of CuSO₄ (2.36 mg, 0.0148 mmol, 0.4 equiv.) and NaAsc (8.7 mg, 0.0439 mmol, 1.2 equiv.) in H₂O (0.3 mL) under argon atmosphere. The reaction mixture was vigorously stirred at room temperature overnight. Then, acetone (30 mL) was added to precipitate the crude product. The crude was then dissolved in water (2 mL) and treated with Quadrasil®MP (80 mg) to remove the residual copper ions. After filtration through a 45 μm sterile filter, the filtrate was passed through a Sephadex® G-15 column and eluted with water. The fractions that could not migrate by TLC elution with DCM/MeOH (1:0.3) were collected. The combined fractions were lyophilized to afford a dark green solid (82 mg, 0.0323 mmol, 87% yield).

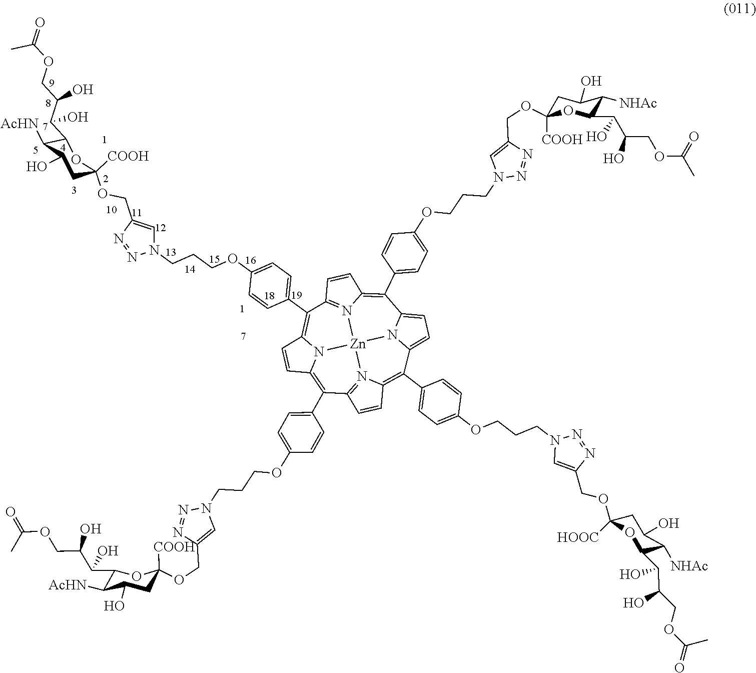

¹H NMR (500 MHz, DMSO-d₆) δ 8.60 (br, 8H, 8×H-_{Porph}), 8.11 (br, 4×H-12), 7.72 (br, 8H, 8×H-18), 6.89 (br, 8H, 8×H-17), 4.18-4.04 (m, 56H, 4×H-4, 4×H-5, 4×H-8, 4×H-9, 4×H-10, 4×H-13, 4×H-15, 12×OH, 4×NH), 3.46-3.33 (m, 8H, 4×H-6, 4×H-7), 2.26 (8H, 4×H-14), 1.88 (br, 28H, 4×NCOCH₃, 4×OCOCH₃, 4×H-3b). ¹³C NMR (126 MHz, DMSO-d₆) δ 174.0 (C═O, NCOCH₃), 172.3 (C═O, C-1, 9-O—Ac), 158.3 (Cq, C-16), 150.3 (Cq, C-19), 135.7 (C-18), 132.2 (C-_{Porph}), 120.7 (Cq, C-_{Porph}), 113.2 (C-17), 73.1 (C-6), 69.6 (C-8), 68.0 (C-4, C-7), 67.0 (C-9), 65.2 (C-15), 58.1 (C-10), 53.1 (C-5), 47.8 (C-13), 41.8 (C-3), 30.1 (C-14), 23.1 (NCOCH₃), 21.3 (OCOCH₃). HRMS (TOF-MS-ESI⁺, m/z): calculated for C₁₂₀H₁₄₀N₂₀O₄₄Zn [M+2H]²⁺ 1313.4228; found 1313.3966.

Compound (013): To a solution of compound 012 (40 mg, 0.0172 mmol, 1 equiv.) and compound 001 (95 mg, 0.274 mmol, 16 equiv.) in DMSO (1.4 mL) was added CuBr·CH₃SCH₃ (3.5 mg, 0.017 mmol, 1 equiv.) under argon atmosphere. After 4 h of microwave irradiation at 80° C., the solution was cooled down and precipitated with DCM (30 mL), and centrifuged. The crude was redissolved in water (2 mL) and treated with Quadrasil®MP (80 mg) to remove the residual copper ions. After filtration through a 45 μm sterile filter, the filtrate was passed through a Sephadex™G-25 column and eluted with water. The fractions that could not migrate by TLC elution with DCM/MeOH were collected. The combined fractions were lyophilized to afford an orange solid (89 mg, 0.0137 mmol, 80% yield).

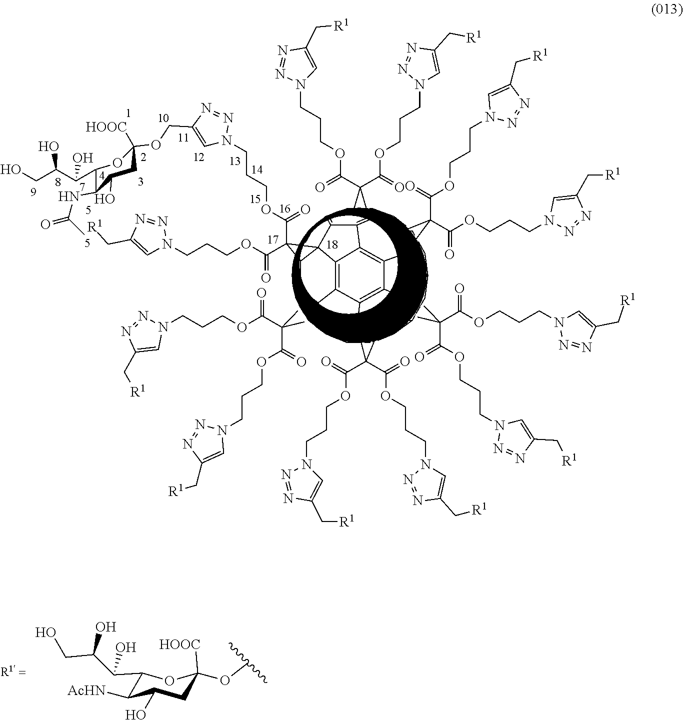

¹H NMR (400 MHz, DMSO-d₆) δ 8.07-7.97 (m, 12H, 12×H-12), 4.67 (s, 12H, 12×H-10a), 4.40 (s, 36H, 12×H-10b, 12×H-15), 4.15 (br, 84H, 12×H-12, 48×OH, 12×NH), 3.69-3.29 (m, 84H, 12×H-4, 12×H-5, 12×H-6, 12×H-7, 12×H-8, 12×H-9), 2.67 (s, 12H, 12×H-3a), 2.15 (s, 24H, 12×H-14), 1.86 (s, 36H, 12×NAc), 1.36 (m, 12H, 12×H-3b). ¹³C NMR (101 MHz, DMSO-d₆) δ 174.0 (C═O, NCOCH₃), 171.9 (C═O, C-1), 164.0 (Cq, C-16), 145.6, 141.5 (C-11, Cₛₚ₂), 125.2 (C-12), 100.7 (C-2), 73.4 (C-6), 72.1, 69.4, 68.2 (C-18, C-8, C-7, C-4), 65.1 (C-15), 63.6 (C-9), 57.8 (C-10), 53.3 (C-5), 47.2 (C-17, C-13), 41.9 (C-3), 29.4 (C-14), 23.2 (NCOCH₃). HRMS (ESI+-MS, m/z): calculated for C₂₈₂H₃₂₈N₄₈O₁₃₂ [M+4H]⁴⁺ 1623.2468; found 1623.2138.

Compound (014): To a solution of compound 012 (22 mg, 0.0096 mmol, 1 equiv.) and compound 002 (60 mg, 0.154 mmol, 16 equiv.) in DMSO (0.8 mL) was added CuBr·CH₃SCH₃ (2 mg, 0.0096 mmol, 1 equiv.) under argon atmosphere. After 4 h of microwave irradiation at 80° C., the solution was cooled down and precipitated with DCM (30 mL), and centrifuged. The crude was redissolved in water (2 mL) and treated with Quadrasil®MP (60 mg) to remove the residual copper ions. After filtration through a 45 m sterile filter, the filtrate was passed through a Sephadex™G-25 column and eluted with water. The fractions that could not migrate by TLC elution with DCM/MeOH (1:0.3, KMnO₄ staining) were collected. The combined fractions were lyophilized to afford an orange solid (55 mg, 0.00785 mmol, 82% yield).

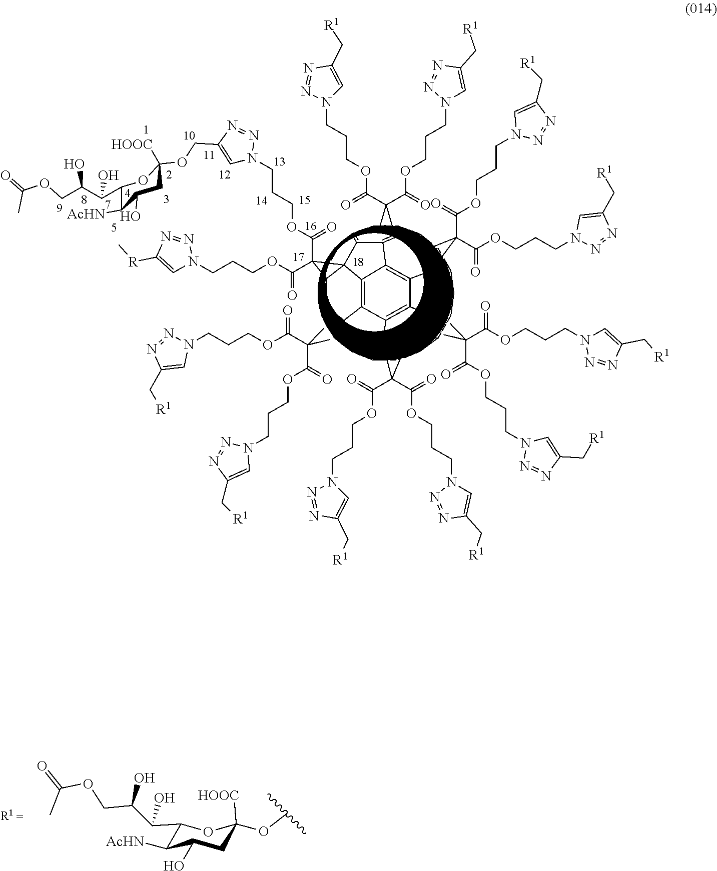

¹H NMR (500 MHz, DMSO-d₆) δ 8.24-8.01 (m, 12H, 12×H-12), 4.94-3.25 (m, 192H, 12×H-4, 12×H-5, 12×H-6, 12×H-7, 12×H-8, 12×H-9, 12×H-10, 12×H-13, 12×H-15), 2.68 (br, 12H, H-3a), 2.16-1.15 (m, 80H, 12×H-3b, 12×H-14, 12×NCOCH₃, 12×OCOCH₃). ¹³C NMR (101 MHz, DMSO-d₆) δ 173.5 (C═O, NCOCH₃), 171.5 (C═O, C-1, 9-O—Ac), 163.57 (Cq, C-16), 145.7, 145.2 (C-11, Cₛₚ₂), 124.5 (C-12), 100.4 (C-2), 72.9 (C-6), 69.4 (C-18, C-8, C-7), 67.8 (C-4), 66.5 (C-9), 64.5 (C-15), 57.64 (C-10), 53.1 (C-5), 46.7 (C-17, C-13), 41.6 (C-3), 29.2 (C-14), 22.9 (NCOCH₃), 21.2 (OCOCH₃). HRMS (ESI+-MS, m/z): calculated for C₃₀₆H₃₄₈N₄₈O₁₄₄ [M+5H]⁵⁺ 1400.6350; found 1400.6368.

### Example 2: AcSA-Derived Glycoclusters are Potent Inhibitors of SARS-CoV-2 Cell Binding and Infectivity

### Materials and Methods

### Generation of UV-Inactivated SARS-CoV-2

SARS-CoV-2 (BavPatl strain, European Virology Archives) was grown in Vero E6 and used at passage 3. 500 μL of passage 3 stock (supernatants of infected cells) was added to a 6-well dish. The 6-well dish was placed in a UV Stratalinker 1800 (Stratagene) without a lid and virus-containing supernatant was exposed to 5000 J of UV irradiation. Virus inactivation was confirmed by adding 10 μL of the UV treated supernatant to a 48-well plate containing 50 000 naïve Vero E6 cells and monitoring infection 48 hours later by indirect immunofluorescence assay.

### Sialic Acid-Coated Surfaces

Gold-coated silicon substrates were first washed with ethanol and cleaned by UV-O treatment (Jetlight) for 15 minutes. The surfaces were then incubated overnight at 4° C. in a biotinylated bovine serum albumin (BBSA) solution (25 μg·mL⁻¹ in PBS, Sigma). After rinsing with PBS, a drop of streptavidin (10 μg·mL⁻¹ in PBS, Sigma) was pipetted onto the BBSA surface for 1 h at 4° C., followed by rinsing with PBS. Finally, the BBSA-streptavidin surfaces were immersed for 1 h in a biotinylated biot-SA (B4) or biot-9-AcSA (B5) solution (10 μg·mL⁻¹ in PBS), followed by a final PBS rinsing. The surfaces showed a homogeneous and stable morphology under repeated scanning and exhibited a thickness of 1.1±0.1 nm. The thickness of the deposited layer was estimated by scanning a small area (1 μm×1 μm) of the surface at high forces to remove the attached biomolecules, followed by imaging larger squares of the same region (5 μm×5 μm) at a lower force.

### Virus Particle Imaging

An 80-μL droplet of virus solution (˜10⁷ particles·mL⁻¹) was deposited on a freshly cleaved mica substrate and incubated at +4° C. for 1 hour. After rinsing 10 times with MilliQ water, the sample was dried for 1 hour at 37° C. AFM imaging was conducted in the PeakForce Tapping mode using PeakForce-Hirs-F-A tips (nominal spring constant 0.4 N·m⁻¹, Bruker) in air. The imaging parameters that were used are: tip oscillation frequency of 1 kHz, maximum peak force of 250 pN; scan rate of 0.25 kHz and displaying 256 pixels per line.

### AFM Tip Functionalization

For AFM tip functionalization, NHS-PEG₂₄-Ph-aldehyde linkers (Broadpharm) were used. AFM tips (MSCT-D probes, Bruker) were immersed in chloroform for 10 min, rinsed with ethanol, dried in a gentle stream of filtered nitrogen, cleaned for 15 min in an ultraviolet radiation and ozone cleaner (JetLight), and immersed overnight in an ethanolamine solution [3.3 g of ethanolamine hydrochloride in 6.6 mL of dimethyl sulfoxide (DMSO)]. The cantilevers were then washed three times with DMSO and three times with ethanol, and dried with nitrogen. Meanwhile, 3.3 mg of NHS-PEG₂₄-Ph-aldehyde linkers were dissolved in 0.5 mL of chloroform. The ethanolamine-coated cantilevers were immersed this solution together with 30 μL triethylamine. After 2 h incubation time, were washed three times with chloroform, dried with nitrogen, and placed in a star conformation (with the tips facing each other) on Parafilm (Bemis NA). 50 μL of S1-subunit protein solution (0.1 mg·mL⁻¹, Genscript Z03501) or UV-inactivated SARS-CoV-2 virions (10⁸ particles mL⁻¹) and 2 μL of freshly prepared NaCNBH₃ solution (6 wt % vol⁻¹ in 0.1 M NaOH(aq)) was pipetted on them and incubated for 1 h at 4° C. Finally, 5 μL of 1 M ethanolamine (pH=8) was added to the drop for 10 min to quench the reaction. After a wash in PBS, the tips were stored in PBS until the experiment.

### Binding Probability (BP) Assays

A Nanoscope Multimode 8 (Bruker) was operated in force-volume (contact) mode to conduct the force spectroscopy experiments on model surfaces (Nanoscope software v9.1). MSCT-D probes (nominal spring constant of 0.03 N m⁻¹) were used to record 5 μm×5 μm maps, with a ramp size of 200 nm, a maximum force of 500 pN, and no surface delay. The sample was scanned using a line frequency of 1 Hz, and 32 pixels per line (32 lines). Both approach and retraction speed were kept constant at 1 μm s⁻¹.

### Screening of Inhibitory Potential

To study the role of sialic acid during the first steps of SARS-CoV-2 binding to cell host surface, FD-curve based AFM was used to compare SARS-CoV-2 binding to SA (Neu5Ac) and 9-AcSA and characterize the binding free-energy landscape of the interaction to 9-AcSA. To mimic exposure of cell-surface glycans in vitro, biotinylated-SA, either biot-9-AcSA (B5) or biot-SA (B4) were immobilized onto streptavidin-coated surfaces and validated by AFM imaging and scratching experiments, revealing a 1.1±0.1 nm thick deposited layer. The interaction between the spike S1 subunit and the SA-coated surfaces was monitored by FD-based AFM.

To study the inhibitory potential of the synthesized (9-Ac)-SA-derived glycoclusters (cf. Example 1-d) on the binding affinity between SARS-CoV-2 and 9-AcSA, binding probabilities (BP) (fraction of curves showing binding events) were measured before and after incubation with different concentrations (0, 1, 10 and 100 μM respectively) of SA-glycoclusters (004, 007, 010 and/or 013) and four 9-AsSA-glycoclusters (005, 008, 011 and/or 014). Three force-volume maps were recorded on three different areas as described previously in the absence of any glycocluster. Thereafter, the tested glycocluster was added to the fluid cell and three maps were recorded for each concentration.

### Dynamic Force Spectroscopy

Dynamic force spectroscopy experiments were performed using a ForceRobot 300 (JPK). Using the same parameters as for the binding probability (BP) assays, with a varying retraction velocity of 0.1, 0.2, 1, 5, 10 and 20 μm s⁻¹. Origin software (OriginLab) was used to display the results in DFS plots, and to generate rupture force histograms for distinct LR ranges and to apply various force spectroscopy models, as previously described (Alsteens, D. et al., Nature Nanotechnology, 2017, Vol. 12, pp. 177-183). These models are used to quantify the energy landscape of this interaction and to extract the kinetic off rate k_{off}, as well as the distance to the transition state xᵤ. For kinetic on-rate analysis, the BP was determined at a certain contact time (t), which is the time the tip is in contact with the surface.

Those data were fitted and K_{D} calculated as described previously (Ranki, C. et al., Proceedings of the National Academy of Sciences of the United States of America, 2008, Vol. 105, pp. 17778-17783). In brief, the relationship between interaction time (τ) and BP is described by the following equation:

$$BP = A \times [ 1 - exp ( \frac{- ( t - t 0 )}{t} ) ] , \qquad ( 1 )$$

wherein A is the maximum BP and t₀ the lag time. Origin software is used to fit the data and extract τ. In the next step, kₒₙ was calculated by the following equation:

$${k}_{on} = \frac{\frac{1}{2} \text{.4} π {r}_{eff}^{3} . {N}_{a}}{3 {η}_{b} τ} \qquad ( 2 )$$

wherein r_{eff} is the radius of the sphere, n_{b} the number of binding partners, and N_{A} the Avogadro constant. The effective volume V_{eff} (4πr_{eff}³) represents the volume in which the interaction can take place. This results in a half-sphere, since only half of the S1 molecules can interact with its corresponding receptor on the substrate.

### Culture of Cell Lines

CHO cells were cultured in Ham's F12 medium (Sigma) supplemented with 10% FBS (Fetal Bovine Serum), penicillin (100 U mL-1), streptomycin (100 g mL⁻¹) (Invitrogen) and 2 mM L-glutamine (Sigma). Cells were incubated at 37° C. with 5% of CO₂ and in an environment saturated in humidity.

### Transduction of Lec2 Cells

Lec2 cells were transduced to express nuclear eGFP as well as cytoplasmic mCherry using H2BeGFP and actin-mCherry-expressing lentiviruses.

Labelling of UV-Inactivated SARS-CoV-2 Virions with Atto488 NHS Ester Dye

200 μL of a 10⁸ particles·mL⁻¹ solution of UV-inactivated SARS-CoV-2 virions were mixed with 10 μL of an Atto488 NHS ester dye (Atto-Tec) (10 mM in dry DMSO) under gentle agitation for 2 hours in 500 μL of acetate buffer (pH 4.5). The free dyes were then eliminated and the fluorescently labelled virions concentrated to the initial concentration through filtration with an Amicon Ultra-0.5 centrifugal filter unit, MWCO 10 kDa (Sigma) (10 minutes centrifugation at 10 000 g for purification, and 2 min at 1 000 g for recovery of the product).

### Virus Binding Assay

A co-culture of CHO and Lec2 (fluorescently labelled with actin-mCherry) was incubated for 1 hour on ice (to prevent internalization) with a 10⁸ particles·mL⁻¹ solution of UV-inactivated SARS-CoV-2 virions coupled with a Atto488 NHS ester dye. The cells were then rinsed three times with PBS and fixed with formaldehyde (4% in PBS for 15 minutes) (Invitrogen, Thermo Fisher Scientific). After a final wash with PBS, cells were imaged with laser scanning confocal microscopy (Zeiss LSM 980) using a 40× water objective. Images were analysed with the Zen blue 2.3 software (Zeiss). A maximum intensity projection was performed to obtain a single image from the z-stack.

### FD-Based AFM and Fluorescence Microscopy on Living Cells

AFM correlative images of CHO cells were acquired using a Bioscope Resolve AFM (Bruker) in PeakForce QNM mode (Nanoscope software v9.2), which is coupled to an inverted epifluorescence microscope (Zeiss Observer Z.1) or confocal laser scanning microscope (Zeiss LSM 900). All the experiments were performed using a 40× oil objective (NA=0.95). Cell images (30-50 μm²) were recorded with forces of 500 pN using PFQNM-LC probes (Bruker) having tip lengths of 17 μm, tip radii of 65 nm and opening angles of 15°. All fluorescence and AFM experiments were realized under cell-culture conditions using the combined AFM and fluorescence microscopy chamber at 37° C. in either Mem α, nucleosides or Ham's F12 culture medium, depending on the cell type (Alsteens, D. et al., Nature Nanotechnology, 2017, Vol. 12, pp. 177-183). Cantilevers were calibrated using the thermal noise method (Hutter, J. L. & Bechhoefer, J., Review of Scientific Instruments, 1993, Vol. 64, pp. 1868-1873), yielding values ranging from 0.08 to 0.14 N/m. The AFM tip was oscillated in a sinusoidal fashion at 0.25 kHz with a 750 nm amplitude. The sample was scanned using a frequency of 0.125 Hz and 128 or 256 pixels per line. AFM images and FD curves were analysed using the Nanoscope analysis software (v1.9, Bruker), Origin, and ImageJ (v1.52e). Individual FD curves detecting unbinding events between the cell surface and S1 or SARS-CoV-2 were analysed using the Nanoscope analysis and Origin software. The baseline of the retraction curve was corrected using a linear fit on the last 30% of the retraction curve. Using the force-time curve, the loading rate (slope) of each rupture event was determined. Optical images were analysed using Zen Blue software (Zeiss).

### Monitoring the Effect of 9-AcSA Porphyrin Addition

The live cell experiments were conducted in the same manner as described above by scanning a suitable area of confluent layers of cells, followed by adding either 10 nM, 100 nM, 1 μM or 10 μM of 9-AcSA porphyrin 011 to the culture medium. The same area was then scanned again to monitor potential changes after addition of the 9-AcSA oligomer.

### Production of SARS-CoV-2 Spike Pseudotyped VSV Virus

pCG1 SARS-CoV-2 spike protein with a C-terminal truncation of 18 amino acid residues plasmid was transfected in HEK-293 cells. The day after, VSV-deltaG virions were transduced in cells with MOI 5 FFU per cell. After 1 hour of incubation at 37° C. with 5% of CO2 and in an environment saturated in humidity, the media was removed and the cells were washed with PBS. The transduced cells were cultured in DMEM supplemented with 5% FBS, 1% penicillin, 1% streptomycin, 2 mM L-Glutamine, 1 mM Na-Pyruvate and NEAA, as well as anti-VSV-G antibody (1:1000). The produced viruses were collected from the media the day after the transduction. Cell debris were cleared by centrifugation (1250×g, 10 min) and with a 0.22 μm filter.

### Infectivity Assay

A549 or A549 ACE2 stable cells (1×10⁴) were seeded in a 96-well plate. The mixture of the pseudotyped virus at MOI 5 and either SA (Neu5Ac), 9-AcSA, SA-porphyrin glycocluster 010 or 9-AcSA-porphyrin glycocluster 011 (as shown on Example 1-d) at increasing concentration (0.001 μM, 0.01 μM, 0.1 μM, 1 μM, 10 μM) were incubated for 15 min at room temperature. The mixture was added in the media and the cells were incubated for 1 hour. The cells were washed with PBS and incubated in fresh cell culture media for 24 hours. The infectivity was monitored via fluorescence and the images were taken with the bioimager device (Amersham Typhoon). The number of infected cells were counted by Fiji.

### Results

### 1. Binding of S1 Subunit to 9-O-Acetylated Sialic Acid (9-O-AcSA)

Strikingly, an about three-fold significant difference between acetylated (B5) and non-acetylated SA (B4) was observed, evidencing a significantly higher avidity of S1 subunit for 9-AcSA over SA.

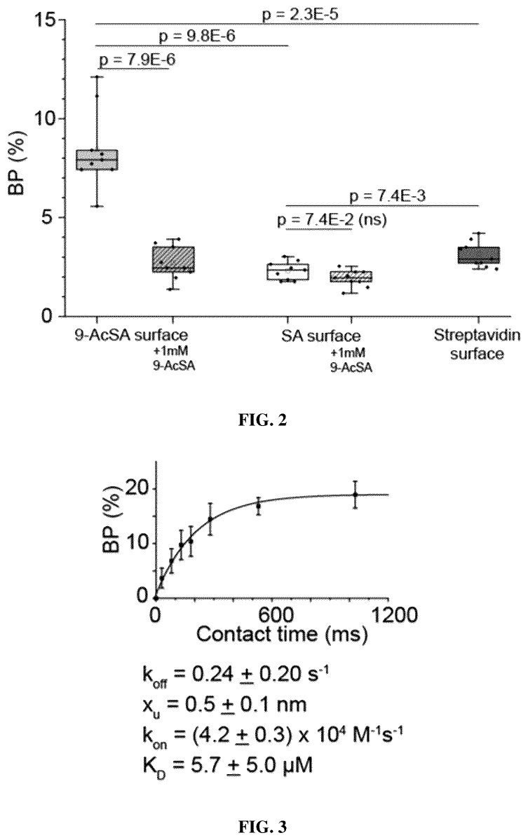

In addition, the specificity of the interaction was confirmed by conducting additional independent control experiments. Both competition assays with free 9-AcSA (FIG. 2) or surface coated with only streptavidin (without SA) resulted in significantly lower binding probability (BP), confirming the specificity of the 9-AcSA interaction with SARS-CoV-2 S1 domain.

### 2. Dynamics of the Interaction Between S1 Subunit and 9-O-AcSA

Binding free-energy landscape of the interaction between S1 and 9-AcSA was characterized using single-molecule dynamic force spectroscopy (DFS). By monitoring the influence of contact time on BP (FIG. 3), the association rate (kₒₙ) was estimated by assuming that the receptor-bond complex can be approximated by a pseudo-fist-order kinetics. By fitting the data with a mono-exponential growth, kₒₙ of (4.2±0.2)×10⁴ M⁻¹s⁻¹ was extracted and the affinity constant (K_{D}) was calculated as the ratio between k_{off} and kₒₙ, resulting in a value of 5.7±5 μM.

Values in the M range correspond to a moderate affinity, supporting the assumption that that SARS-CoV-2 uses glycans such as 9-AcSA as a first moderate-affinity foothold on the host cell surface, facilitating subsequent strong binding to ACE2 receptor.

### 3. Binding at the SARS-CoV-2 Virion Level

Non-replicating SARS-CoV-2 particles (i.e., native SARS-CoV-2 virions inactivated through UV radiation) were used to evaluate the physiological relevance of the probed interaction. The binding of these non-replicating SARS-CoV-2 particles to 9-AcSA was evaluated, by grafting the whole virions onto the AFM tip. The interaction was probed at moderate (1 μm·s⁻¹) and fast (20 μm·s⁻¹) pulling speed, and the DFS plot were reconstructed and overlaid with the data obtained with the purified S1 domain.

A good agreement was observed between the data collected with the purified S1 domain only or with the full virions. Both the different rupture forces at the single-molecule level were very close but also the kinetic parameters. At the virion level, higher forces compatible with multiple contacts were also recorded.

### 4. Validation of the Interaction on Living Cells

The interaction was next validated directly on living cells by probing the interaction between either purified S1 or full non-replicative virions and a co-culture of unlabelled CHO cells, naturally expressing sialic acids (SA), and Lec2 cells (fluorescently labelled with a nuclear protein H2B-GFP and actin-mCherry). To evidence the role of SA as surface receptors, we performed laser-scanning confocal microscopy on a co-culture of CHO and Lec2 cells and observed that UV-inactivated SARS-CoV-2 virions (fluorescently labelled with Atto488-NHS dye) preferentially bind to SA-expressing CHO cells. Then, using AFM tips functionalized with either S1 glycoprotein or the full virions, a confluent monolayer of co-cultured CHO and Lec2 cells was scanned by AFM using conditions to propagate both cell types.

CHO cells showed a significantly higher density of adhesion events (˜9% for S1, ˜13% for SARS-CoV-2, FIG. 13a, n=10 cells for S1 and n=12 cells for SARS-CoV-2), whereas Lec2 cells displayed only a sparse distribution of these events (˜3% for S1, ˜5% for SARS-CoV-2, FIG. 13b, n=10 cells for S1 and n=12 cells for SARS-CoV-2), confirming the establishment of specific SARS-CoV-2 bonds to SA on living cells.

### 5. Inhibition of SARS-Cov-2 Binding Using SA- and AcSA-Glycoclusters

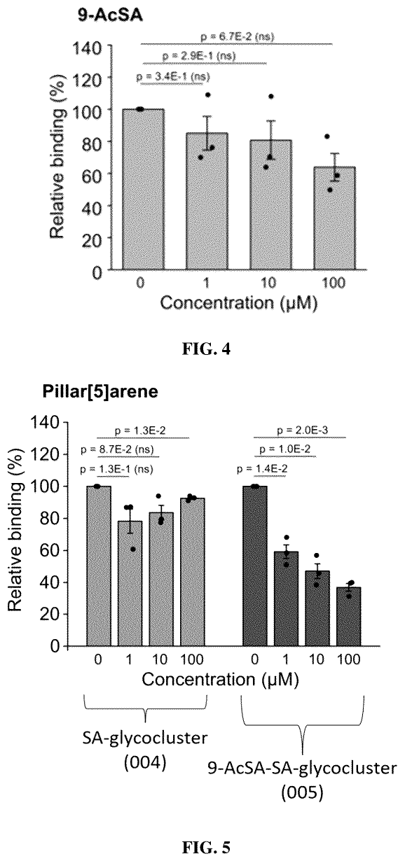

The eight glycoclusters decorated either with the SA or 9-AcSA were tested for their blocking properties using our SMFS approach and full non-replicative SARS-CoV-2 particles. First, the inhibiting capacity of the monovalent, commercially available 9-acetylated sialic acid (9-AcSA) (Carbosynth) was evaluated at 0, 1, 10 and 100 μM (FIG. 4). Only a slight reduction of 20-30% in the binding frequency (BF) over the explored concentration range was observed. This result was expected as it was determined earlier that even the more active AcSA (compared to SA) displays only a moderate affinity for the SARS-Cov-2 spike, as evidenced also in Example 2.2.

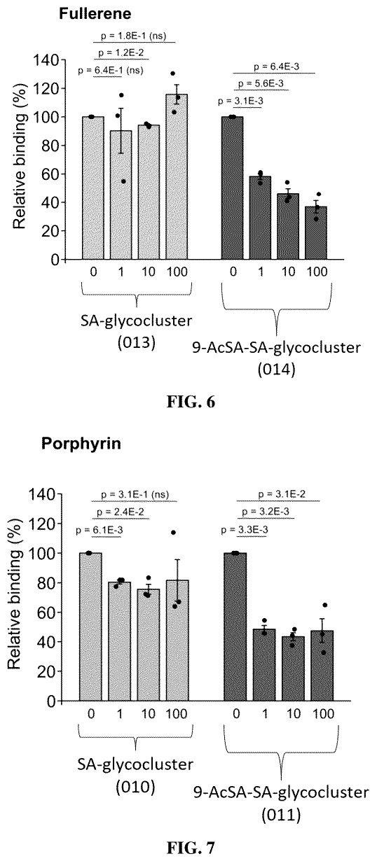

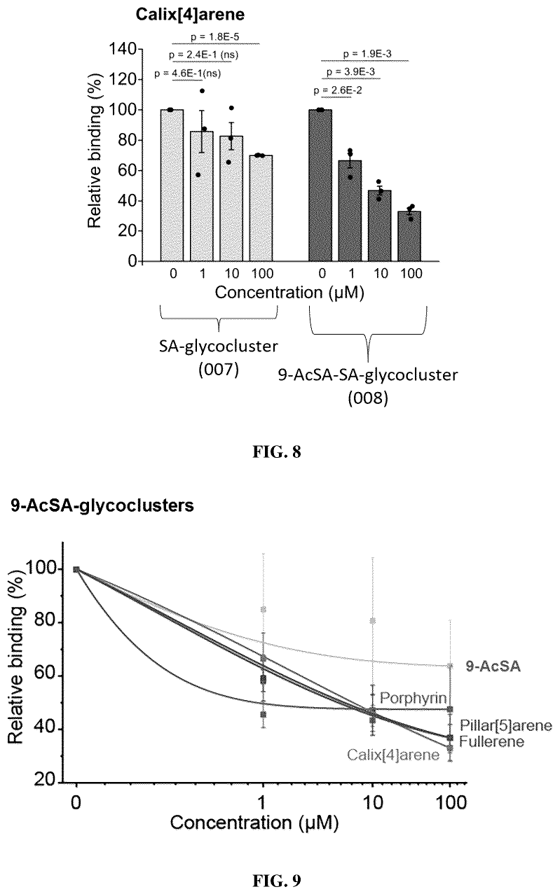

The four glycoclusters either functionalized with SA or 9-AcSA were compared (FIGS. 5 to 8). The relative binding frequency (BF) plots show either no or slight inhibition for the SA-glycoclusters, whereas a progressive and effective reduction in the BF is observed for 9-AcSA-glycoclusters. Moreover, a 50% reduction around 10 μM was observed for all 9-AcSA-clusters. The 9-AcSA-derived porphyrin 011 seems the most efficient inhibitor, reaching a reduction of about 50% already at 1 μM (FIGS. 7 and 9).

Therefore, the Applicants surprisingly evidenced that a plurality of AcSA covalently linked to porphyrins, pillararenes, calixarenes and fullerenes leads to potent inhibition of the SARS-CoV-2 despite the low affinity of the free AcSA. Thus, the glycoclusters of the invention may be used in the treatment and/or prevention of an infectious disease such as COVID-19.

### 6. Characterization of the Inhibition of an AcSA-Derived Porphyrin

As the first screening (Example 2.5 herein) pointed out the porphyrin-based glycocluster (compound 011) as the most efficient 9-AcSA-derived glycocluster for SARS-CoV-2 anti-binding, its binding potency was characterized in more detail.

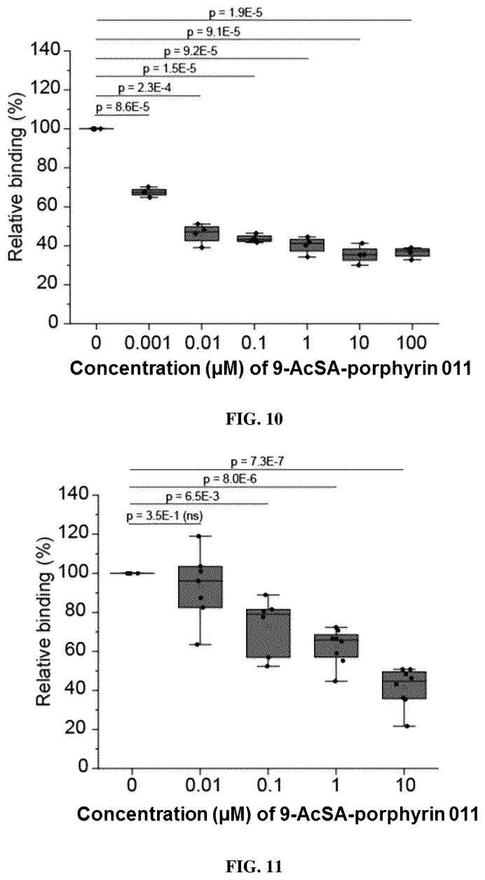

Stunningly, it was found that the 9-AcSA-porphyrin 011 leads to a reduction of more than 50% of the probed interactions between SARS-CoV-2 and 9-AcSA already at 10 nM and follows an exponential decay of as a function of the concentration (FIG. 10).

The inhibition potential on living cells was probed and this trend was confirmed also under physiological relevant condition (FIG. 11).

Therefore, the Applicants unexpectedly evidenced that a plurality of AcSA covalently linked to porphyrins lead to especially significant inhibition of the SARS-CoV-2 even in low concentrations, despite the low affinity of the free AcSA. Thus, the AcSA-porphyrin-based glycoclusters of the invention may be used in the treatment and/or prevention of an infectious disease such as COVID-19.

### 7. Characterization of the Neutralization Properties of an AcSA-Derived Porphyrin

After evidencing the anti-adhesive capacities (Examples 2.5 and 2.6), it was of interest of evaluating the neutralization properties of the AcSA-porphyrin glycocluster 011, i.e., its capacity to prevent the entry of the virus and thus the infection. A robust virus infectivity assay was used, which can be carried out at low biosafety conditions. Briefly, propagation-incompetent G-deleted vesicular stomatitis virus (VSV) trans-complemented with the SARS-CoV-2 spike protein and encoding for a GFP reporter protein (VSV-SARS-CoV-2) was used on A549 cells and A549 cells transduced with the ACE2 receptor with or without interfering molecules at various concentrations. A549-ACE2 were infected with a MOI of 5 of the VSV-SARS-CoV-2. Infectivity was monitored by measuring the GFP fluorescence in the cells 24 h post-infection. While A549 cells are not infected by the VSV-SARS-CoV-2, overexpression of ACE2 strongly enhanced infection. Next, the infectivity assay was performed while incubating the VSV-SARS-CoV-2 with SA, 9-AcSA, SA-porphyrin 10 and AcSA-porphyrin 11.

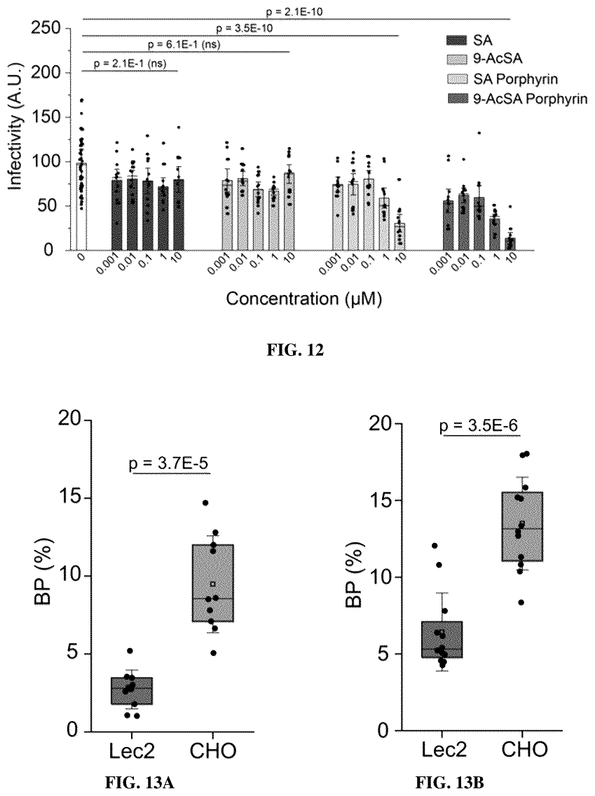

As expected, both SA and 9-SA did not significantly reduce VSV-SARS-CoV-2 infectivity (FIG. 12). Moreover, while SA-porphyrin 010 did not provide significant reduction of VSV-SARS-CoV-2 infectivity, we observed that the 9-AcSA-porphyrin 011 significantly reduced VSV-SARS-CoV-2 infectivity, with an estimated IC₅₀ in the range 0.1-1 μM (FIG. 12).

This cell-based assay confirms the previous results on the effect of sialic acids on SARS-CoV-2 binding and further evidences that effective inhibition of the virus binding to its receptor by a glycocluster of the invention leads to a significant drop in infectivity.

### 8. Comparison of 9-AcSA Porphyrin Binding to Different SARS-CoV-2 Lineages

In order to evaluate whether the SARS-CoV-2 mutations observed in the last circuiting variants have an influence on the efficacy of inhibition of the virus binding to its receptor, an affinity measurement of the 9-AcSA porphyrin 011 to the S1 domain from either the original Wuhan strain or the latest Omicron strain was performed. Briefly, the S1 domain of the spike protein from one or the other strain was covalently attached to a bio-layer interferometry sensor and the binding of 9-AcSA porphyrin 011 was evaluated.

No loss of affinity was observed, the analysis giving affinity constants of the order of 141±4 nM for the Wuhan strain and 100±3 nM for the Omicron strain.

These results confirm the efficacy of acetylated sialic acids glycoclusters against the original strain of SARS-CoV-2, but also against the Omicron strain despite it having appeared nearly 2 years after the original haplotype and comprising a large number of mutations.

1-15. (canceled)

16. A glycocluster comprising at least two acetylated-sialic acids covalently linked to a macrocycle,
wherein said macrocycle is selected from porphyrins, pillararenes, calixarenes and fullerenes; and
wherein each acetylated-sialic acid is independently selected from 4-O-acetylated-sialic acid, 7-O-acetylated-sialic acid, 8-O-acetylated-sialic acid and 9-O-acetylated-sialic acid.

17. The glycocluster according to according claim 16, wherein said glycocluster comprises at least four acetylated-sialic acids covalently linked to said macrocycle.

18. The glycocluster according to according claim 16, wherein each acetylated-sialic acid is independently selected from 7-O-acetylated-sialic acid and 9-O-acetylated-sialic acid.

19. The glycocluster according to according claim 18, wherein each acetylated-sialic acid is 9-O-acetylated-sialic acid.

20. The glycocluster according to according claim 16, wherein said macrocycle is selected from porphyrins, preferably selected from [Zn(tetraphenylporphyrin)] and tetraphenylporphyrin.

21. The glycocluster according to according claim 16, wherein said glycocluster further comprises at least one angiotensin-converting enzyme 2 (ACE2) binding inhibitor; preferably the angiotensin-converting enzyme 2 (ACE2) binding inhibitor is selected from ACE2 binding inhibitor peptides, ACE2 binding inhibitor proteins and anti-ACE2 antibodies or antigen-binding fragments thereof.

22. The glycocluster according to according claim 16, wherein said glycocluster is a compound of formula (I), (Ia), (II), (III) or (IV): 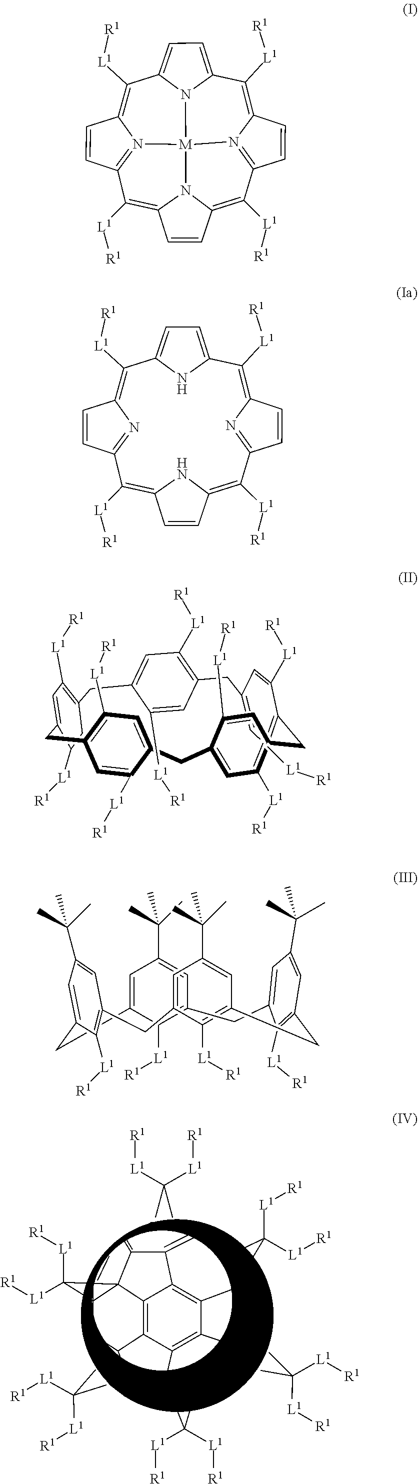
or a pharmaceutically acceptable salt and/or solvate thereof;
wherein:
each L¹ is independently:
a single bond or
a linker selected from alkyl, heteroalkyl, alkenyl, alkynyl, aryl, alkylaryl, arylalkyl, heteroaryl, heteroarylalkyl and alkylheteroaryl; wherein said alkyl, heteroalkyl, alkenyl, alkynyl, aryl, alkylaryl, arylalkyl, heteroaryl, heteroarylalkyl or alkylheteroaryl optionally comprises at least one coupling product;
each R¹ is independently selected from acetylated-sialic acids and ACE2 binding inhibitors; provided that at least two R¹ are acetylated-sialic acids; and
M is a metal cation.

23. The glycocluster according to according claim 22, wherein each L¹ is a linker selected from alkyl, heteroalkyl, alkylaryl, arylalkyl, heteroarylalkyl and alkylheteroaryl; wherein said alkyl, heteroalkyl, alkylaryl, arylalkyl, heteroarylalkyl or alkylheteroaryl comprises one or two coupling products; preferably at least one of said coupling products is triazolyl.

24. The glycocluster according to according claim 22, wherein each L¹-R¹ is selected from —(CH₂)ₘ—R^{C}—(CH₂)ₙ—R¹, —O—(CH₂)ₘ—R^{C}—(CH₂)ₙ—R¹, —C(O)—(CH₂)ₘ—R^{C}—(CH₂)ₙ—R¹, —C(O)O—(CH₂)ₘ—R^{C}—(CH₂)ₙ—R¹, -phenyl-(CH₂)ₘ—R^{C}—(CH₂)ₙ—R¹ and -phenyl-O—(CH₂)ₘ—R^{C}—(CH₂)ₙ—R¹;
wherein R^{C} is a coupling product, preferably triazolyl; m and n are independently an integer ranging from 1 to 8, preferably ranging from 1 to 4; and each R¹ is as defined above.

25. The glycocluster according to according claim 22, wherein each R¹ is 9-O-acetylated-sialic acid.

26. The glycocluster according to according claim 25, wherein said glycocluster is a compound of formula (011), (005), (008) or (014): 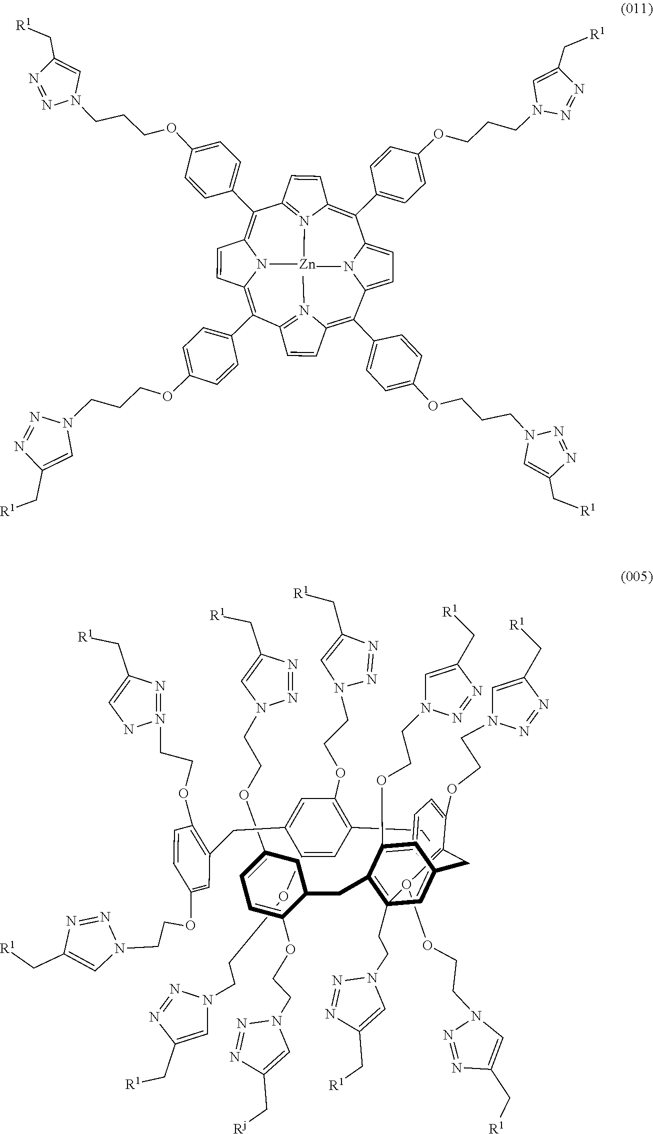 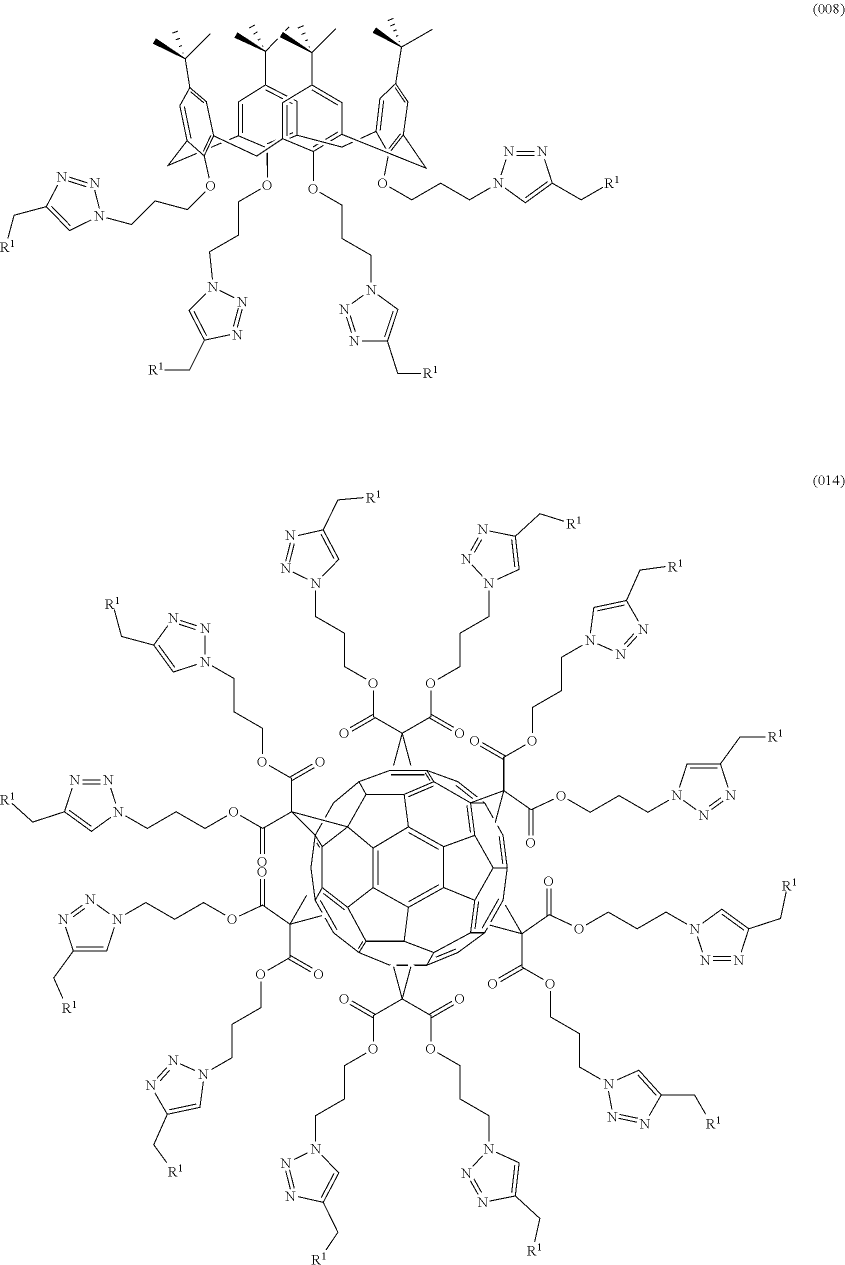
or a pharmaceutically acceptable salt and/or solvate thereof;
wherein each R¹ is of formula (9-AcSA) 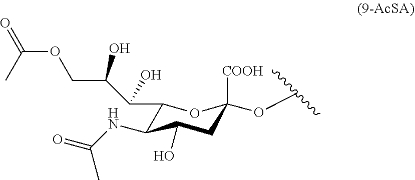
wherein the wavy line represents the point of attachment of R¹ to the glycocluster.

27. A pharmaceutical composition comprising a glycocluster according to claim 16 and at least one pharmaceutically acceptable carrier.

28. A medicament comprising the glycocluster according claim 16 or a pharmaceutical composition comprising said glycocluster and at least one pharmaceutically acceptable carrier.

29. A method of treating and/or preventing an infectious disease, preferably a coronavirus or picornavirus infection, more preferably a SARS-CoV-2 infection, in a subject in need thereof, comprising administering to said subject a therapeutically effective amount of the glycocluster according to claim 16 or a pharmaceutical composition comprising said glycocluster and at least one pharmaceutically acceptable carrier.

30. A process for manufacturing a glycocluster according claim 16, comprising a step of coupling of each of said acetylated-sialic acids with said macrocycle; preferably a coupling wherein the reaction between a terminal alkyne and an azide results in the formation of a triazolyl group.


SEQUENCE LISTING (4 sequences)

SEQ ID NO: 1 - protein, 23 aa, synthetic construct
EEQAKTFLDKFNHEAEDLFYQSS

SEQ ID NO: 2 - protein, 7 aa, synthetic construct
LGKGDFR

SEQ ID NO: 3 - protein, 36 aa, synthetic construct
EEQAKTFLDKFNHEAEDLFYQSSLASWNYNTNITEE

SEQ ID NO: 4 - protein, 31 aa, synthetic construct
EEQAKTFLDKFNHEAEDLFYQSSGLGKGDFR